(19) United States
(12) Patent Application Publication
(10) Pub. No.: US 2008/0044472 A1
(43) Pub. Date: Feb. 21, 2008

(54) Photoresponsive Hydrogels

(76) Inventors: Antonio A. Garcia, Chandler, AZ (US); Rohit Rosario, Daly City, CA (US); John Devens Gust JR., Mesa, AZ (US); Mark A. Hayes, Gilbert, AZ (US); Manuel Marquez, Midlothian, VA (US); Zhibing Hu, Denton, TX (US); Tong Cai, Denton, TX (US)

Correspondence Address: NEEDLE & ROSENBERG, P.C., SUITE 1000, 999 PEACHTREE STREET, ATLANTA, GA, 30309-3915, US

(21) Appl. No.: 10/587,229

(22) PCT Filed: Jan. 24, 2005
(86) PCT No.: PCT/US05/02317

§ 371 Date: Oct. 31, 2007

Related U.S. Application Data
(60) Provisional application No. 60/538,687, filed on Jan. 23, 2004.

(51) Int. Cl. A61K 31/407; A61K 31/711; A61K 45/00; A61K 9/14; A61P 29/00

(52) U.S. Cl. 424/484; 424/93.1; 514/409; 514/44

(57) ABSTRACT

Disclosed are photoresponsive hydrogels. The compositions disclosed herein can be prepared by polymerizing a hydrogel precursor and a spiropyran. The properties of the disclosed compositions can be changed by exposure to light, pH, and temperature. Methods of using the disclosed compositions to deliver pharmaceutical actives are also disclosed.

### I. CROSS-REFERENCE TO RELATED APPLICATIONS

This application claims benefit of priority to U. S. Provisional Application No. 60/538,687, filed Jan. 23, 2004, which is incorporated by reference herein in its entirety.

### II. BACKGROUND

Recent advances in materials science have stimulated tremendous activity in immobilizing or encapsulating cells, nutraceuticals, flavors, pharmaceuticals, and other materials for a host of applications (see e.g., Willaert and Baron, Rev. Chem. Eng. 12:5-205 (1996); Lanza, et al., Principles of Tissue Engineering (Academic Press, San Diego, 2000); Chaikof, Annu. Rev. Biomed. Eng. 1:103-127 (1999); Bodeutsch, et al., Plant Cell Reports 20:562-566 (2001); Decamps, et al., Aiche Journal 50:1599-1605 (2004); Bergers and Hanahan, Nature Biotech 19:20-21 (2001); Desai, et al., Biomol. Eng. 17:23-26 (2000); Park, et al., Biotechnol. Adv. 18:303-319 (2000); Orive, et al., Trends Biotechnol. 22:87 (2004); Green, et al., Biotechnology and Bioengineering 49:535-543 (1996); Gref, et al., Science 263:1600 (1994); Mills and Needham, Expert Opin. Ther. Patents 9:1499-1513 (1999); Gouin, Trends Food Sci. Technol. 15:330-347 (2004); Re, Drying Technology 16:1195-1236 (1998); Dziezak, Food Technology—Chicago 42:136-151 (1988); Gibbs, et al., Int. J Food Sci. Nutr. 50:213-224 (1999); Dinsmore, et al., Science 298:1006 (2002)). In general, the goals of such research is to construct a composition or device that allows independent control of the capsule size and surface chemistry, and the permeability of select agents. A particularly attractive goal is the ability to stimulate the release of encapsulated materials on demand and reversibly.

With encapsulated cells, the advantages of encapsulation include increased biocatalytic efficiency and lifetime, as well as increased ease of handling and separation from the products. Nutrients and waste pro ducts can be rapidly exchanged, yet the cells are protected against shear stresses, which could suppress their output. With nutrients, drugs, or flavors, the advantages of encapsulation include the possibility of protecting the materials from chemical degradation and releasing the materials at the optimal time or location for more efficient delivery.

Despite a host of recent advances, however, current approaches to encapsulation generally suffer from expense, toxicity, or a lack of control of the capsule architecture at the scale of about 10 to about 100 nm. Although the capsules themselves can be about 10's of micrometers or larger in size, the pore sizes can be in the nanometer range. Controlling the nanoscale pore sizes and shapes can be desirable to achieve selective permeability of cells, macromolecules, and other kinds of agents (e.g., nutraceuticals or pharmaceuticals). Therefore, what are needed are compositions that can be used to encapsulate a wide variety of materials and that can have their properties controlled by various means. The compositions and methods disclosed herein meet these needs.

### III. SUMMARY

In accordance with the purposes of the disclosed materials, compounds, compositions, articles, and methods, as embodied and broadly described herein, the disclosed subject matter, in one aspect, relates to compounds and compositions and methods for preparing and using such compounds and compositions. In another aspect, disclosed herein are photoresponsive hydrogels and methods for preparing compositions thereof. The disclosed subject matter also related to methods of using the disclosed compositions to deliver pharmaceutical actives.

The advantages described below will be realized and attained by means of the elements and combinations particularly pointed out in the appended claims. It is to be understood that both the foregoing general description and the following detailed description are exemplary and explanatory only and are not restrictive.

### IV. BRIEF DESCRIPTION OF THE FIGURES

The accompanying figures, which are incorporated in and constitute a part of this specification, illustrate several aspects described below.

FIG. 1 is an arrangement of several images of colloidosomes. Panel (a) is an optical micrograph (brightfield) of yeast cells in a colloidosome in aqueous broth solution. Panel (b) is a scanning electron micrograph of empty colloidosomes in vacuum, demonstrating the morphology of the pores (Dinsmore, et al., Science 298:1006 (2002)). Panel (c) is a further magnification of one region of the colloidosome, showing the pores. Experiments in solution showed that molecules can indeed permeate the pores (Id.).

FIG. 2 (top) is an illustration of the closed and open states of a spiropyran (SP) compound, before and after protonation. FIG. 2 (bottom) is a schematic of the photoresponsive gel chemistry along with approximate mole % of each monomer: PNIPAAm (n=100); N,N′-methylenebisacrylamide (n′=2); open, protonated spiropyran (n″=1).

FIG. 3 is a graph of hydrodynamic radius distributions ƒ(Rₕ)) of PNIPAAm-SP nanoparticles under dark in deionized water with different synthesis conditions. The LLS measurements were at a 60° scattering angle. Batch 1 SP was dissolved in deionized water. Batch 2 SP was dissolved in about pH 8 NaOH / deionized water solution.

FIG. 4 is a pair of graphs showing the hydrodynamic radius (ƒ(Rₕ)) of PNIPAAm-spiropyran (SP) nanoparticles, measured by dynamic light scattering at a scattering angle of 600. The top graph shows that the PNIPAAm-SP nanoparticles swell as light conditions change from dark to UV and to visible irradiation at 21° C. The bottom graph shows that the PNIPAAm-SP nanoparticles change their size in response to different pH at 31° C. in the dark.

FIG. 5 is a graph showing the thermally responsive behavior of PNIPAAm-SP nanoparticles. The top graph shows the average hydrodynamic radius Rₕ of PNIPAAM-SP nanoparticles at different pH value as a function of temperature under dark. The bottom graph shows the temperature dependent average hydrodynamic radius Rₕ of PNIPAAm-SP nanoparticles under different light conditions as a function of temperature in deionized water.

FIG. 6 is a photograph of 8 weight % PNIPAAm-SP nanoparticles with different pH at 21° C. Panel (a) is at about pH 3, panel (b) is deionized water, panel (c) is at about pH 9.

FIG. 7 is a graph of 8 weight % PNIPAAm-SP nanoparticles in water under dark at different temperatures. From bottom to top, the lines in the graph are at 27° C., 31° C., 32° C., 33° C., and 34° C., respectively.

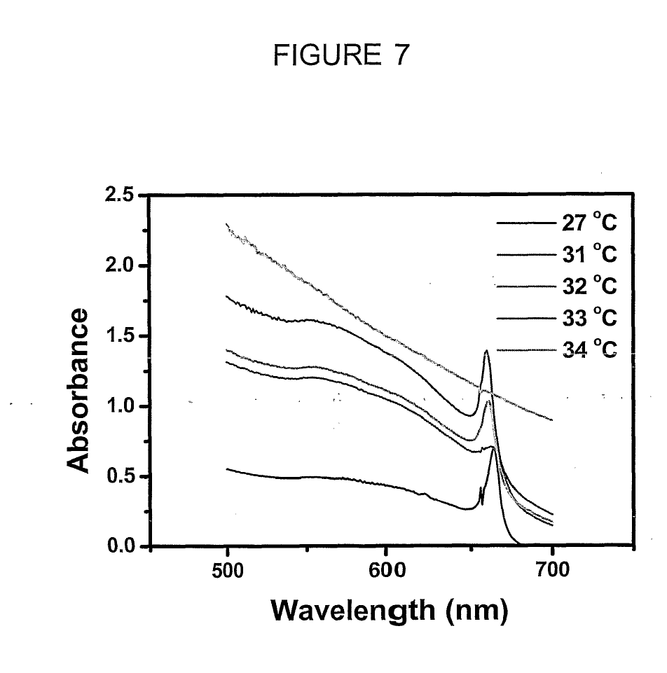

FIG. 8 is a schematic of the mechanism of ICAM-1 AS-ODN treatment.

FIG. 9 is a schematic of the use of photoactive nanogels as gene carriers.

### V. DETAILED DESCRIPTION

The materials, compositions, articles, devices, and methods described herein may be understood more readily by reference to the following detailed description of specific aspects of the disclosed subject matter, and methods and the Examples included therein and to the Figures and their previous and following description.

Before the present materials, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed subject matter pertains. The references disclosed are also individually and specifically incorporated by reference herein for the material contained in them that is discussed in the sentence in which the reference is relied upon.

1. General Definitions

In this specification and in the claims that follow, reference will be made to a number of terms, which shall be defined to have the following meanings:

As used in the specification and the appended claims, the singular forms “a,” “an,” and “the” include plural referents unless the context clearly dictates otherwise. Thus, for example, reference to “a compound” includes mixtures of two or more such compounds, reference to “an agent” includes mixtures of two or more such agents, reference to “the nanoparticle” includes mixtures of two or more such nanoparticles, and the like.

Throughout the specification and claims, the word “comprise” and variations of the word, such as “comprising” and “comprises,” means “including but not limited to,” and is not intended to exclude, for example, other additives, components, integers or steps.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and claims to parts by weight of a particular element or component in a composition or article denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “hydrogel” is meant a composition comprising a polymeric dispersed phase in aqueous dispersion medium (i.e., continuous phase). The dispersed phase can be an amorphous network of polymers or discrete hydrogel precursors. When the dispersed phase comprises nanoscale hydrogel precursors of from about 1 to about 1000 nm, the hydrogel can be termed a “nanogel.” When the dispersed phase comprises microscale particles of from about 1 to about 5 micrometer, the hydrogel can be termed a “microgel.” The term “colloidosome” can also be used to describe a particular hydrogel where the dispersed phase comprises spherical, densely-packed nano-or microscale particles. As used herein, the term “hydrogel” is meant to include and is used interchangeably with the terms “nanogels,” “microgels,” “colloidosomes,” and mixtures thereof. The terms are also used herein to refer to the polymeric dispersed phase alone, in the absence of the aqueous continuous phase.

2. Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Also, as used herein “substitution” or “substituted with” is meant to encompass configurations where one substituent is fused to another substituent. For example, an alkyl group substituted with an aryl group can mean that the aryl group is bonded to the alkyl group via a single sigma bond and also that the aryl group and alkyl group are fused, e.g., two carbons of the alkyl group are shared with two carbons of the aryl group.

“A,” “A¹,” “A²,” “A³,” and “A⁴” are used herein as generic symbols to represent various specific substituents. These symbols can be any substituent, not limited to those disclosed herein, and when they are defined to be certain substituents in one sentence does not mean that, in another sentence, they cannot be defined as other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkylalcohol” specifically refers to an alkyl group that is substituted with one or more hydroxyl groups, as described below, and the like. When “alkyl” is used in one sentence and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group may be defined as —OA where A is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, or thiol as described herein.

The term “cycloalkenyl” as used herein is a non-aromatic carbon-based ring composed of at least three carbon atoms and contains at least one double bound, e.g., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The terms “amine” or “amino” as used herein are represented by the formula NAA¹A², where A, A¹, and A² can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “ester” as used herein is represented by the formula —OC(O)A or —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula AC(O)A¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —^{l}SiAA¹ A², where A, A¹, and A² can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfo-oxo” as used herein is represented by the formulas —S(O)A (sulfoxide), —S(0)₂A (sulfonyl), —OS(O)₂A (sulfone), or —OS(O)₂OA, where A can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

“R¹,” “R²,” “X” and “L” as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is(are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Reference will now be made in detail to specific aspects of the disclosed materials, compounds, compositions, components, devices, articles, and methods, examples of which are illustrated in the following description and examples, and in the figures and their previous and following description.

A. General Compositions

Disclosed herein are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a molecule is disclosed and a number of modifications that can be made to a number of substituents are discussed, each and every combination and permutation that are possible are specifically contemplated unless specifically indicated to the contrary. Thus, if a class of substituents A, B, and C are disclosed as well as a class of substituents D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Certain materials, compounds, compositions, and components disclosed herein can be obtained commercially or can be readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

1. Specific Compositions

Disclosed herein are compositions that are or can form hydrogels (e.g., nanogels and macrogels) and colloidosomes when, e.g., mixed with water. The compositions comprise photoactive particles with unusual and well-controlled properties, which have a variety of uses, such as encapsulating devices and delivery vehicles (e.g., olidonucleotide delivery devices).

In response to environmental stimuli such as temperature or pH, the disclosed hydrogel compositions can change their volume by several orders of magnitude (Li and Tanaka, Annu. Rev. Mat. Sci. 22:243 (1992)) enabling applications in controlled drug release (Hoffman, Adv. Drug Delivery Rev. 54:3 (2002); Siegel and Firestone, Macromolecules 21:3254-3259 (1988); Jeong, et al., Nature 388:860-862 (1997); Wang, et al., Nature 397:417-420 (1999); Gehrke, Adv. Polym. Sci. 110:81 (1993) and sensing (Osada, et al., Nature 355:242-244 (1992); Hu, et al., Science 269:525-527 (1995)). Further, as disclosed herein, the disclosed hydrogen compositions can comprise spiropyrans (either mixed with the hydrogel precursor component of the hydrogel or bonded to the hydrogel precursor). These spiropyrans can act as triggering molecules that respond to external light with rapid changes in conformation, polarity, and charge. This, in turn, can trigger large changes in the charge, polarity, and degree of swelling of the particles, resulting in the change in permeability of nanogel, microgel, hydrogel, or colloidosome. Also, as hydrogels, the disclosed compositions can, in one aspect, be readily used for biomedical applications because of their ability to simulate biological tissues (Peppas, Hydrogels in Medicine and Pharmacy (CRC Press, Boca Raton, Fla., 1987); Peppas and Langer, Science 263:1715 (1994)).

In one aspect, disclosed herein in a composition produced by the process comprising polymerizing a hydrogel precursor with a spiropyran. In another aspect, the disclosed compositions comprise a hydrogel precursor and a spiropyran. In another aspect, the disclosed compositions can be produced by the process comprising reacting a hydrogel precursor comprising at least one hydroxyl group and/or carboxylic acid group with a spiropyran comprising a group capable of reacting with the hydroxyl group or carboxylic acid group.

The compositions disclosed herein can be on the micrometer or nanometer scale and can have unique properties that can be changed due to differences in, for example, light, pH, and temperature conditions.

The development of photo-responsive gels has been the subject of intensive investigation since the discovery of the volume phase transition in polymer gels (Tanaka, Phys. Rev. Lett. 40:820 (1978)). This is because light triggered volume phase transition is convenient, environmentally friendly, and can be remotely controlled (Suzuki and Tanaka, Nature 346:345-347 (1990)). Ultraviolet light was used to initiate an ionization reaction in the gel, creating internal osmotic pressure (Irie and Kunwatchakun, Macrom. Rapid Comm. pp. 2476-2480; Mamada, et al., Macromolecules 23:1517 (1990)). The phase transition of gels was also induced by directly heating the network polymers by visible light (Suzuki and Tanaka, Nature 346:345-347 (1990)) or infrared radiation (Zhang, et al., J. Chem. Phys. 102:551 (1995)). A light-modulation gel has been prepared that imitates the behavior of pigment cells (Akashi, et al., Adv. Mater. 14:1808 (2002)). Light triggered anisotropic bending has been found in azobenzene liquid-crystalline gels (Ikeda, et al., Adv. Mater. 15:201 (2003)). An azobenzene cross-linked hydrogel which contains an embedded crystalline colloidal array has been prepared for potential application in recordable and erasable memories (Hirakura, et al., Biomacromolecules 5:1804-1809 (2004); Sumaru, et al., Macromolecules 37:4949-4955 (2004)). Proton dissociation of spirobenzopyran-functionalized poly(N-isopropylacrylamide) in aqueous solution has been studied using light irradiation as a trigger (Kameda, et al., Langmuir 20:9315-9319 (2004); Sumaru, et al., Macromolecules 37:7854-7856 (2004); Garcia, et al., J. Phys. Chem. A 104:6103-6107 (2000)). Photoresponsive nanogels were prepared by the self-assembly of spiropyrane-bearing pullulan (Rosario, et al., Langmuir 18:8062-8069 (2002)).

While other researchers have observed the photoresponsiveness of polymers containing pendant spiropyran groups or hydrogel with pendant azobenzene or leucohydroxide groups, no work has been described on hydrogels with pendant spiropyran groups. One reason for this is that spiropyrans are not generally very soluble in water.

As disclosed herein, spiropyrans such as spiropyran (SP) are incorporated into the hydrogel precursor component or nanoparticles (e.g., PNIPAAm nanoparticles) to prepare photoactive hydrogels. The hydrogels disclosed herein (e.g., spiropyran-NIPA hydrogels) are unlike azobenzene and leucohydroxides in that upon UV irradiation, spiropyran is converted to a zwitterionic form in aqueous solution. This leads to more versatile charge-electric field applications at different pH values along with unique interactions with biological molecules. Further, the synthetic routes disclosed herein have the advantages of avoiding the addition of surfactant for biomedical compatibility, and the formation of monodisperse samples with controlled sizes. As a result, these particles can be sensitive to multiple stimuli including light, pH, and temperature, and they can also have a monodisperse size distribution that enable them to self-assemble into 3D ordered structures.

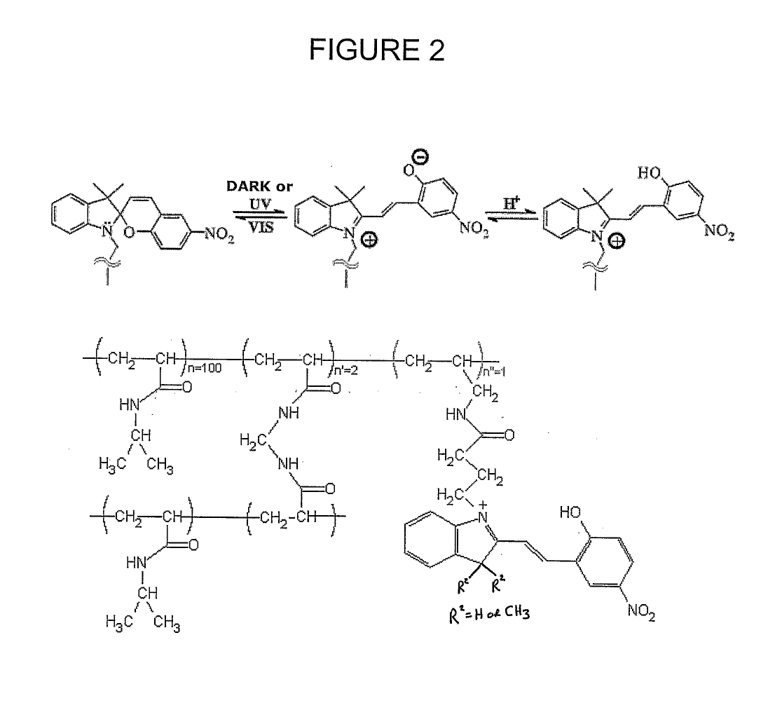

As disclosed herein, when spiropyrans such as those described herein can be copolymerized with hydrogel precursor monomers like N-isopropylacrylamide (NIPAAm), poly-NIPAAm (PNIPAAm) nanogel particles can be created that are both thermally- and photonically-responsive (FIG. 2 bottom). At temperatures above their lower critical solution temperature (LCST) the nanogels disclosed herein contract sharply and expel water from their structure, and when cooled they rehydrate and expand. Also, depending upon their polymerization conditions, the gels can also be made to contract and expand using different wavelengths of light or by alternating between visible light and darkness. In fact, UV irradiated particles (260 nm radius) were shown to be smaller than those under visible irradiation (520 nm radius). The fact that nanogels undergo light-induced physical changes allows them to be custom-tailored as highly-specific delivery vehicles for gene therapy. For example, by exposing the disclosed hydrogels to various conditions (e.g., light, pH, temperature), one can specifically control the delivery of encapsulated oligonucleotides.

2. Size

As disclosed herein, the size of the hydrogel compositions can be adjusted by treatment with different light conditions (e.g., visible light, UV light, dark), temperature, and pH. The size of the compositions can also be adjusted, if desired, by a variety of other procedures including, but not limited to, extrusion, filtration, sonication, homogenization, employing a laminar stream of a core of liquid introduced into an immiscible sheath of liquid, extrusion under pressure through pores of defined size, and similar methods. The foregoing techniques, as well as others, are discussed, for example, in Mayer, et al., Biochim. Biophys. Acta. 858:161-168 (1986); Hope, et al., Biochim. Biophys. Acta. 812:55-65 (1985); Mayhew, et al., Methods in Enzymology 149:64-77 (1987). The disclosures of these publications are incorporated by reference herein in their entireties.

In general, the hydrogel compositions disclosed herein can have at least one dimension (e.g., hydrodynamic radius, diameter, length, width, height, etc.) less than about 1 micrometer (1000 nanometers (nm)), less than about 750 nm, less than about 500 nm, less than about 250 nm, less than about 100 nm, and/or less than about 10 nm. In one aspect, the particles disclosed herein can have at least one dimension greater than about 1 micrometer, greater than about 750 nm, greater than about 500 nm, greater than about 250 nm, greater than 100 nm, and/or greater than about 10 nm. In still other aspects, the disclosed particles can have at least one dimension in the range of from about 1 to about 10 nm, from about 10 to about 100 nm, from about 100 to about 200 nm, from about 100 to about 300 nm, from about 200 to about 300 nm, from about 300 to about 400 nm, from about 100 to about 400 nm, from about 200 to about 400 nm, from about 400 to about 500 nm, from about 100 to about 500 nm, from about 200 to about 500 nm, from about 300 to about 500 nm, from about 500 to about 600 nm, from about 100 to about 600 nm, from about 200 to about 600 nm, from about 300 to about 600 nm, from about 400 to about 600 nm, from about 600 to about 700 nm, from about 100 to about 700 nm, from about 200 to about 700 nm, from about 300 to about 700 nm, from about 400 to about 700 nm, from about 500 to about 700 nm, from about 700 to about 800 nm, from about 100 to about 800 nm, from about 200 to about 800 nm, from about 300 to about 800 nm, from about 400 to about 800 nm, from about 500 to about 800 nm, from about 600 to about 800 nm, from about 800 to about 900 nm, from about 100 to about 900 nm, from about 200 to about 900 nm, from about 300 to about 900 nm, from about 400 to about 900 nm, from about 500 to about 900 nm, from about 600 to about 900 nm, from about 700 to about 900 nm, from about 900 to about 1000 nm, from about 100 to about 1000 nm, from about 200 to about 1000 nm, from about 300 to about 1000 nm, from about 400 to about 1000 nm, from about 500 to about 1000 nm, from about 600 to about 1000 mu, from about 700 to about 1 000 nm, and/or from about 800 to about 1000 nm. In another aspect, the particles disclosed herein can have at least one dimension of about 1, 2, 5, 10, 15, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750,775, 800, 825, 850, 875, 900, 925, 950, 975, and/or 1000 nm, where any of the stated values can form an upper or lower endpoint as appropriate.

3. Spiropyran

The compositions disclosed herein contain one or more spiropyrans. The spiropyran can act as a triggering molecule or switch in the disclosed compositions because it responds to external stimulus (light) with rapid changes in its properties such as conformation, polarity, and/or charge. These changes in turn can trigger large changes in the charge, polarity, and degree of swelling of the disclosed compositions.

Spiropyrans are one of the most useful and well studied classes of photoactive molecules (Bertelson, in Photochromism, G. H. Brown, Ed. (Wiley-Interscience, New York, 1971)). In solution and irradiated with visible light, the majority of spiropyrans are colorless (or nearly so). Under these conditions the spiropyran exists in a nonpolar or “closed” spiro form that absorbs light predominantly in the ultraviolet region (FIG. 2 top). When exposed to UV light, or in polar solvents in the dark, SP can undergo an isomerization wherein the spiro linkage is severed, resulting in a highly polar “open” form that is colored (typically absorbing near 530 nm) and highly fluorescent (Bunker, et al., Nano Letters 3:1723-1727 (2003)). Interfacial force microscopy was used to directly determine that the large light-induced change in polarity of spiropyran-coated surfaces was due to the reversible formation of a positively charged surface under UV irradiation or in the dark.

In one aspect, a spiropyran that is suitable for the compositions and methods disclosed herein can undergo a change in its properties (e.g., polarity, charge, or conformation) upon exposure to visible light, red light, or blue light. For example, the spiropyran can undergo a change in its properties upon exposure to light of from about 400 nm to about 800 nm. In some specific examples, the spiropyran can undergo a change in its properties upon exposure to light of about 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434,435, 436, 437, 438, 439, 440, 441, 442, 443, 444, 445, 446, 447, 448, 449, 450, 451, 452,453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, and/or 800 nm, where any of the stated values can form an upper or lower endpoint as appropriate.

In another aspect, a spiropyran that is suitable for the compositions and methods disclosed herein can undergo a change in its properties upon exposure to ultraviolet light. For example, the spiropyran can undergo a change in its properties upon exposure to light of from about 1 nm to about 300 nm. In some specific examples, the spiropyran can undergo a change in its properties upon exposure to light of about 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44,45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, and/or 300 nm, where any of the stated values can form an upper or lower endpoint as appropriate.

In another aspect, mixtures of spiropyrans can be used that under go changes unpon exposure to any of the decribed wavelengths of light.

Suitable spiropyrans that can be used in the disclosed compositions include, but are not limited to, spiropyran compounds having the following Formula I: 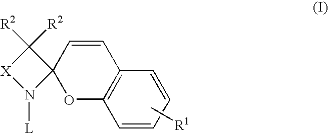
wherein X is a substituted or unsubstituted, C1to C4, alkyl or alkenly group; R¹ is H, alkyl, alkenyl, alkoxy, aryl, halide, hydroxyl, amino, nitro, silyl, sulfo-oxo, sulfonylamino, ether, ester, carboxylic acid, or thiol group; each R² is, independently of each other, H, alkyl, alkenyl, alkoxy, aryl, halide, hydroxyl, amino, nitro, silyl, sulfo-oxo, sulfonylamino, thiol, ether, ester, carboxylic acid, or together each R² substituent forms a keto group, a cyclic alkyl group, a cyclic alkenyl group, or an aryl group; and L is H or linker, wherein the linker is capable of forming at least one bond with a hydrogel or hydrogel precursor (e.g., a alkenyl group). In one aspect, the spiropyran can comprise at least one alkenyl group. In another aspect, X is a fused aryl group.

In one aspect, described herein are compositions comprising a compound represented by Formula I. In another aspect, described herein are compositions prepared by or with compounds represented by Formula I.

Compounds represented by Formula I can be optically active or racemic. The stereochemistry at the various chiral centers in Formula I can vary and will depend upon the spatial relationship between the substituents on that carbon. In one aspect, the stereochemistry at a chiral carbon shown in Formula I is S. In another aspect, the stereochemistry at a chiral carbon shown in Formula I is R. Using techniques known in the art, it is possible to vary the stereochemistry at each chiral carbon shown in Formula I. While such enantioselective and enantiospecific techniques typically provide the one isomer, the presence of a minor amount of the other isomer can sometimes occur.

a) R¹ Substituent

As disclosed herein, the R¹ substituent can comprise an alkyl, alkenyl, alkoxy, aryl, halide, hydroxyl, amino, nitro, silyl, sulfo-oxo, sulfonylamino, ether, ester, carboxylic acid, or thiol group, or any combination thereof. The R1 substituent can be in the ortho, meta, or para position.

In one aspect, the R¹ substituent can comprise an electron withdrawing group such a nitro, sulfonyl, or halogenated alkyl group. In another aspect, R¹ can be an alkyl group such as methyl, ethyl, n-propyl, i-propyl, n-butyl, i-butyl, s-butyl, t-butyl, n-pentyl, i-pentyl, neopentyl, hexyl, heptyl, octyl, nonyl, decyl, dodecyl, tetradecyl, hexadecyl or derivatives thereof. In other examples, the R¹ substituent can be a substituted alkyl, such as an alkylalcohol or halogenated alkyl. Examples of suitable alkylalcohols for a R¹ substituent include, but are not limited to, hydroxymethyl, hydroxylethyl, hydroxypropyl, dihydroxypropyl, hydroxybutyl, dihydroxybutyl, hydroxypentyl, dihydroxypentyl, hydroxyhexyl, dihydroxyhexyl, 3-methyl-2-hydroxybutanyl, 2-methyl-3-hydroxypentyl, and derivatives thereof. Examples of suitable halogenated alkyl groups for a R¹ substituent include, but are not limited to, chloro- or bromomethyl, chloro- or bromoethyl, chloro- or bromopropyl chloro- or bromobutyl, and derivatives thereof.

In still another aspect, a R¹ substituent can comprise an alkoxy group, such as a methoxy, ethoxy, methoxymethyl, ethoxymethyl, propoxy, isopropoxy, butoxy, tertbutoxy, neopentoxy, and the like.

In one specific examples, the R¹ substituent is a nitro group.

b) R² Substituent

As disclosed herein, each R² substituent can comprise, independent of the other R² substituent, H, alkyl, alkenyl, alkoxy, aryl, halide, hydroxyl, amino, nitro, silyl, sulfo-oxo, sulfonylamino, thiol, ether, ester, carboxylic acid, or together each R² substituent forms a keto group, a cyclic alkyl group, a cyclicalkenyl group, or an aryl group, or any combination thereof.

In one aspect, R² can be an alkyl group such as methyl, ethyl, n-propyl, i-propyl, n-butyl, i-butyl, s-butyl, t-butyl, n-pentyl, i-pentyl, neopentyl, hexyl, heptyl, octyl, nonyl, decyl, dodecyl, tetradecyl, hexadecyl or derivatives thereof. In other examples, the R² substituent can be a substituted alkyl, such as an alkylalcohol or halogenated alkyl. Examples of suitable alkylalcohols for a R¹ substituent include, but are not limited to, hydroxymethyl, hydroxylethyl, hydroxypropyl, dihydroxypropyl, hydroxybutyl, dihydroxybutyl, hydroxypentyl, dihydroxypentyl, hydroxyhexyl, dihydroxyhexyl, 3-methyl-2-hydroxybutanyl, 2-methyl-3-hydroxypentyl, and derivatives thereof. Examples of suitable halogenated alkyl groups for a R² substituent include, but are not limited to, chloro- or bromomethyl, chloro- or bromoethyl, chloro- or bromopropyl chloro- or bromobutyl, and derivatives thereof.

In still another aspect, a R² substituent can comprise an alkoxy group, such as a methoxy, ethoxy, methoxymethyl, ethoxymethyl, propoxy, isopropoxy, butoxy, tertbutoxy, neopentoxy, and the like.

In one aspect, both R² substituents can form together a keto group, a cyclicalkynyl group (e.g., a substituted or unsubstitued cyclopropyl, cyclobutyl, cyclopentyl, cyclohexyl, or cycloheptyl group), or a cyclicalkenyl group (e.g., a substituted or unsubstitued cyclopentenyl cyclopentenyl, cyclopentadienyl, cyclohexenyl, cyclohexadienyl), and the like. Further examples include, heterocyloalkenyl groups, such as pyrrolino, imidazolino, pyrazolino, azirino, oxirenyl, azepine, pyranyl, and the like. Still further examples include heterocycloalkyl groups, i.e., a cycloalkyl group wherein one of the carbon atoms is substituted with, for example, oxygen, sulfur, or nitrogen. Examples of suitable heterocycloalkyl groups for a pair of R² substituents include, but are not limited to, tetrahydrofuranyl, tetrahydrofurfuryl alcohol, tetrahydrofurfurylamine, tetrahydrofurfuryl acetate, tetrahydropyranyl, pyrrolidino, piperidino, piperazino, morpholino and thiomorpholino, thiomorpholino-1-oxide, thiomorpholino-1,1-dioxide, 1,4-dioxane, oxepaneyl, and the like.

In one specific example, at least one or both R² groups are methyl groups.

c) X Moiety

As disclosed herein, X is a substituted or unsubstituted, C1 to C4, alkyl or alkenly group. That is, in Formula I, the nitrogen containing ring can be a 4-, 5-, 6-, or 7-membered, saturated or unsaturated ring. This X moiety of the ring can be substituted or unsubstituted. In one aspect, the X group can be substituted with alkyl, alkenyl, alkoxy, aryl, halide, hydroxyl, amino, nitro, silyl, sulfo-oxo, sulfonylamino, ether, ester, carboxylic acid, or thiol group, or any combination thereof.

Further, as mentioned earlier, X can be substituted with an aryl group in a manner that results in a fused ring structure. For example, X can share two carbon atoms with an aryl group, including non-heteroaryl and heteroaryl groups. Suitable non-heteroaryl groups with which X can be substituted (or fused) include, but are not limited to, phenyl, halophenyl, methylphenyl, dimethylphenyl, ethylphenyl, propylphenyl, hydroxyphenyl, aminophenyl, carboxyphenyl, styrenyl, indenyl, naphthyl, biphenyl, anthracenyl, fluorenyl, phenanthrenyl, tosyl, and the like, and derivatives thereof. Suitable heteroaryl groups include, but are not limited to, pyridinyl, furanyl, thiophenyl, thiazolyl, isothiazolyl, triazolyl, imidazolyl, isoxazolyl, pyrrolyl, pyrazolyl, pyrimidino, pyrazino, pyridazino, benzofuranyl, isobenzofuranyl, benzothiazolyl, benzoisothiazolyl, benzotriazolyl, indolyl, isoindolyl, 3-indole-sulfate, indo-2-carboxylic acid, indolinyl, indolizinyl, benzocazolyl, quinolyl, isoquinolyl, quinazolinyl, benzimidazolyl, benzisoxazolyl, benzothiophenyl, dibenzofuran, imidazolyl, acridinyl, phenothiazinyl, carbazolyl, and benzodiazepin-2-one-5-yl, and the like, and derivatives thereof.

In another example, X can be substituted with or fused with (i.e., share two carbon atoms with) a cyclicalkyl group (e.g., a cyclopentyl or cyclohexyl), a cyclicalkenyl group (e.g., cyclopentenyl, cyclopentadienyl, or cyclohexenyl), a heterocycloalkyl group (e.g., tetrahydrofuranyl, tetrahydrofurfuryl alcohol, tetrahydrofurfurylamine, tetrahydrofurfuryl acetate, tetrahydropyranyl, pyrrolidino, piperidino, piperazino, morpholino and thiomorpholino, thiomorpholino-1-oxide, thiomorpholino-1,1-dioxide, 1,4-dioxane, oxepaneyl), or a heterocycloalkenyl group (e.g., pyrrolino, imidazolino, pyrazolino, azirino, oxirenyl, azepine, pyranyl).

d) Linker

As disclosed herein, L of Formula I is a linker. That is, L is a chemical moiety that is capable of forming a bond with a Formula I to the hydrogel precursors disclosed herein. When L is present in Formula I, it can attach to the hydrogel precursor at any location.

L can be of varying lengths, such as from 1 to 12 atoms in length. For example, L can be from 1, 2, 3, 4, 5, 6, 7, 8, 9, 10, 11, or 12 atoms in length, where any of the stated values can form an upper or lower end point as appropriate. L can be substituted or unsubstituted. When substituted, L can contain substituents attached to the backbone of L or substituents embedded in the backbone of L. For example, an amine substituted linker L can contain an amine group attached to the backbone of L or a nitrogen in the backbone of L. Suitable moieties for L include, but are not limited to, substituted or unsubstituted, branched or unbranched, alkyl, alkenyl, or alkynyl groups, ethers, esters, polyethers, polyesters, polyalkylenes, polyamines, heteroatom substituted alkyl, alkenyl, or alkynyl groups, cycloalkyl groups, cycloalkenyl groups, heterocycloalkyl groups, heterocycloalkenyl groups, and the like, and derivatives thereof.

In one aspect, when L is present in Formula I, L can be a C1 to C8 branched or straight-chain alkyl, such as methyl, ethyl, n-propyl, i-propyl, n-butyl, i-butyl, s-butyl, t-butyl, n-pentyl, i-pentyl, neopentyl, or hexyl. In a specific example, L can be —(CH₂)ₙ-, wherein n is from 1 to 4.

In another aspect, when L is present in Formula I, L can be a C1 to C8 branched or straight-chain alkoxy such as a methoxy, ethoxy, n-propoxy, i-propoxy, n-butoxy, i-butoxy, s-butoxy, t-butoxy, n-pent oxy, i-pentoxy, neopentoxy, or hexoxy.

In still another aspect, when L is present in Formula I, L can be a C2 to C8 branched or straight-chain alkyl, wherein one or more of the carbon atoms is substituted with oxygen (e.g., an ether) or an amino group. For example, suitable linkers (L) can include, but are not limited to, a methoxymethyl, methoxyethyl, methoxypropyl, methoxybutyl, ethoxymethyl, ethoxyethyl, ethoxypropyl, propoxymethyl, propoxyethyl, methylaminomethyl, methylaminoethyl, methylaminopropyl, methylaminobutyl, ethylaminomethyl, ethylaminoethyl, ethylaminopropyl, propylaminomethyl, propylaminoethyl, methoxymethoxymethyl, ethoxymethoxymethyl, methoxyethoxymethyl, methoxymethoxyethyl, and the like, and derivatives thereof.

In yet another aspect, when L is present in Formula I, L can be a C1 to C8 amine or amide. For example, L can be a methyl amide (i.e., a urea linker in Formula I), ethyl amide or amide, propyl amide or amine, butyl amide or amine, pentyl amide or amine, hexyl amide or amine, heptyl amide or amine, or octyl amide or amine. In one specific example, L is a butyl amide or allyl amide. In another example, L can be —(CH₂)ₘC(O)NH(CH₂)ₙCH═CH₂, wherein m is from 1 to 12 and n is from 0 to 12. In yet another example, m can be 3 and n can be 1.

### e) SPECIFIC EXAMPLES

One specific example of a spiropyrans that is suitable for use in the disclosed hydrogel compositions is the spiropyran of Formula II: 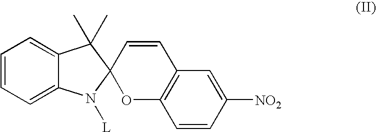
wherein L is as described above, e.g., L is linker capable of forming at least one bond with a hydrogel precursor.

It should be understood that the spiropyrans disclosed herein can be converted (via changes in light, pH, temperature, etc.) to a different form. For example, the compound of Formula II in the presence of dark or UV light and a proton source will exist as the following isomer (Formula III).

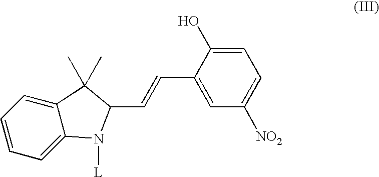

Thus, reference herein to compounds of Formula I or II are meant to refer and identify both the closed and open isomer forms of these molecules as illustrated in Formula III.

J) Preparation

The spiropyrans disclosed herein can be prepared by methods known in the art. For example, recent syntheses and characterization studies were performed by Gust, et al., (Langmuir 19:8801-8806 (2003)), which is incorporated by reference herein for its teachings of preparing and characterizing spiropyrans. These studies have led to spiropyran molecules such as those of Formulae I-III, which have enough water solubility to perform aqueous polymerizations with hydrogel precursors like NIPA and can lead to the hydrogel compositions disclosed herein. Photoactive spiropyrans can be also be prepared and studied in oil/water systems (Garcia, et al., J. Phys. Chem. A 104:6103-6107 (2000)) and covalently bound to surfaces (Rosario, et al., Langmuir 18:8062-8069 (2002); Rosario, et al., Langmuir 19:8801-8806 (2003); Rosario, et al., Proceedings of Spie: 4807. Physical Chemistry of Interfaces and Nanomaterials, Zhang and Wang, Eds. (2002) pp. 197, which are incorporated by reference herein for their teachings of preparing and characterizing spiropyrans), leading to hydrophobic property changes upon irradiation with UN and visible light.

g) Amounts

The amount of the spiropyran in the hydrogel compositions disclosed herein can be any amount, but will typically be from about 1 to about 20 weight % of the composition. For example, the spiropyran can be present in an amount of from about 1 to about 20 weight %, from about 1 to about 15 weight %, from about 1 to about 10 weight %, from about 1 to about 7 weight %, from about 1 to about 3 weight %, or from about 1 to about 2 weight % of the compositions disclosed herein. In other examples, the spiropyran can be present in an amount of about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 weight %, wherein any of the stated values can form an upper or lower endpoint a appropriate.

4. Hydrogel Precursor

The hydrogel precursor is one or more monomers, that are the same or different and that can be used to prepare a hydrogel. The hydrogel precursor can be of a single type, e.g., capable of forming a homopolymer, or more than one type, e.g. capable of forming a copolymer. Copolymers are suitable for the hydrogel compositions disclosed herein can be random, graft, or block polymers. The disclosed hydrogel precursors can form polymeric particles on the nanoscale or microscale.

Examples of suitable hydrogel precursors are monomers that can be used to prepare polyesters, such as terephthalate based polymers, polyesteramides, cellulose esters, polyurethanes, polycarbonates, epoxy resins, polyamides, vinyl polymers (e.g., polystyrene, polyethylene, polypropylene, polybutylene, polyacrylonitrile, poly(methyl)metacrylate, polyacrylamide, polyacrylic acid), hydroxypropylcellulose (HPC), hydroxymethylpropylcellulose, and hyaluronic acid (HA) and other polysacrylate nanoparticles or any mixture thereof.

In one aspect, the hydrogel precursor can comprise a compound having at least one alkenyl group. For example, the hydrogel precursor can comprise acrylonitrile, acrylic acid, acrylamide, or methacrylic acid. In another example, the hydrogel precursor can comprise a substituted acrylamide. In yet another example, the hydrogel precursor can comprise an N-alkyl substituted acrylamide. In still other examples, the hydrogel precursor can comprise N-methylacrylamide, N-ethylacrylamide, N-propyllacrylamide, or N-isopropylacrylamide. In another specific example, the hydrogel precursor comprises hydroxypropylglucose (forming HPC) (Pelton and Chibante, Coll. Surf 20:247 (1986)).

a) Amounts

The amount of the hydrogel precursor in the compositions disclosed herein can be any amount, but will typically be an amount capable of forming a polymer that is from about 99 to about 80 weight % of the hydrogel composition. For example, the hydrogel precursor can be present in an amount capable of forming a polymer that is from about 80 to about 99 weight %, from about 80 to about 97 weight %, from about 80 to about 95 weight %, from about 80 to about 93 weight %, from about 80 to about 90 weight %, or from about 80 to about 85 weight % of the hydrogel compositions disclosed herein. In other examples, the hydrogel precursor can be present in an amount capable of forming a polymer that is about 80, 80.1, 80.2, 80.3, 80.4, 80.5, 80.6, 80.7, 80.8, 80.9, 81.0, 81.1, 81.2, 81.3, 81.4, 81.5, 81.6, 81.7, 81.8, 81.9, 82.0, 82.1, 82.2, 82.3, 82.4, 82.5, 82.6, 82.7, 82.9, 82.9, 83.0, 83.1, 83.2, 83.3, 83.4, 83.5, 83.6, 83.7, 83.8, 83.9, 84.0, 84.1, 84.2, 84.3, 84.4, 84.5, 84.6, 84.7, 84.8, 84.9, 85.0, 85.1, 85.2, 85.3, 85.4, 85.5, 85.6, 85.7, 85.9, 85.9, 86.0, 86.1, 86.2, 86.3, 86.4, 86.5, 86.6, 86.7, 86.8, 86.9, 87.0, 87.1, 87.2, 87.3, 87.4, 87.5, 87.6, 87.7, 87.8, 87.9, 88.0, 88.1, 88.2, 88.3, 88.4, 88.5, 88.6, 88.7, 88.8, 88.9, 89.0, 89.1, 89.2, 89.3, 89.4, 89.5, 89.6, 89.7, 89.8, 89.9, 90.0, 90.1, 90.2, 90.3, 90.4, 90.5, 90.6, 90.7, 90.8, 90.9, 91.0, 91.1, 91.2, 91.3, 91.4, 91.5, 91.6, 91.7, 91.8, 91.9, 92.0, 92.1, 92.2, 92.3, 92.4, 92.5, 92.6, 92.7, 92.8, 92.9, 93.0, 93.1, 93.2, 93.3, 93.4, 93.5, 93.6, 93.7, 93.8, 93.9, 94.0, 94.1, 94.2, 94.3, 94.4, 94.5, 94.6, 94.7, 94.8, 94.9, 95.0, 95.1, 95.2, 95.3, 95.4, 95.5, 95.6, 95.7, 95.8, 95.9, 96.0, 96.1, 96.2, 96.3, 96.4, 96.5, 96.6, 96.7, 96.8, 96.9, 97.0, 97.1, 97.2, 97.3, 97.4, 97.5, 97.6, 97.7, 97.8, 97.9, 98.0, 98.1, 98.2, 98.3, 98.4, 98.5, 98.6, 98.7, 98.8, 98.9, or 99.0 weight % of the hydrogel composition, where any of the stated values can form an upper or lower endpoint as appropriate.

b) Crosslinking

The hydrogels compositions can also contain varying degrees of cross-linking. Thus, in the methods disclosed herein for preparing the dislosed compositions, the methods can further comprise the addition of a crosslinking agent. For example, the hydrogels can comprise from about 0 to about 10 weight %, from about 0 to about 7 weight %, from about 0 to about 5 weight %, from about 0 to about 3 weight %, from about 0 to about 2 weight %, from about 0 to about 1 weight %, or from about 0 to about 0.5 weight percent of a crosslinking agent. In other examples, the hydrogel can comprise about 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 weight % of a crosslinking agent, where any of the stated values can form an upper or lower endpoint as appropriate.

Examples of suitable crosslinking agents that can be used in the hydrogel precursor disclosed herein include, but are not limited to, glycerine, diethanolamine, triethanolamine, tetrahydroxyethylethylenediamine, trimellitic anhydride, benzenetricarboxylic acids and esters thereof, pyromellitic dianhydride, trimethylolpropane, 1,1,1-tris(hydroxymethyl)ethane, pentaerythritol, tartaric acid, citric acid, gallic acid, pyrogallol, divinylbenzene, triallylamine, divinylimidazole, N,N′-divinylethyleneurea, products of the reaction of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin, and allyl or vinyl ethers of polyhydric alcohols, for example 1,2-ethanediol, 1,4-butanediol, diethylene glycol, trimethylolpropane, sorbitan and sugars such as sucrose, glucose, mannose, pentaerythritol triallyl ether, macrodiisocyanates (MDIC), and allyl ethers of sugars such as sucrose, glucose, or mannose. In one specific example, the crosslinking agent can be methylenebisacrylamnide.

5. Incorporation of Spiropyran and Hydrogel Precursor

In one aspect, the disclosed composition can comprise a hydrogel and a spiropyran. The spiropyran can be incorporated or admixed with the hydrogel or hydrogel precursor. In another aspect, the spiropyran can be bonded to the hydrogel or hydrogel precursor. In yet another aspect, the spiropyran can be bonded to one or more hydrogel precursors, which are then used to prepare a hydrogel. By “bonded,” or other forms of the word such as “bonds” or “bound,” is meant any type of interaction between atoms in which there is a donation, acceptance, or sharing of electrons, or an electrostatic interaction. Some examples of bonds that can exists in the compositions disclosed herein include, but are not limited to, covalent bonds, sigma bonds, pi bonds, ionic bonds, dative bonds, and multi-center bonds. In one specific example, the disclosed spiropyrans are covalently bonded to the hydrogel or one or more hydrogel precursors.

Spiropyrans such as spiropyran can be bonded to the hydrogel by copolymerization. For example, spriopyran can be co-polymerized with poly-N-isopropylacrylamide (PNIPAAm) to form hydrogel nanoparticles using the precipitation polymerization method. Poly-N-isopropylacrylamide (PNIPAAm) gel is one of the most used thermally responsive gels. It undergoes a drastic volume change from a swollen state for T<T_{c} to a collapsed state for T>T_{c}, where T_{c} is the lower critical solution temperature, approximately 34° C. (Hirotsu, et al., J. Chem. Phys. 87:1392-1395 (1987)). The value of T_{c} can be increased by copolymerization of polar molecules or decreased by copolymerization of nonpolar molecules.

PNIPAAm is compatible with cells and has already -been used for cell cultures (Kwon, et al., J. Biomed. Mater. Res. 50:82 (2000)). Recent experiments by the Hu group revealed that PNIPAm nanoparticles triggered lesser inflammatory and fibrotic responses than well known biomaterials poly-L-lactic acid (PLA) nanoparticles (Weng, et al., J. Biomater. Sci., Polymer Ed. 15:1167 (2004)). Usually nanoparticles are made by emulsion polymerization with a surfactant. In practice, complete removal of a surfactant from the resulted hydrogel precursors is required for biomedical application. But this removal can be difficult. In contrast, an advantage of the proposed method is to use SP instead. After polymerization, SP can be covalently bonded into the PNIPAAm network and change its role from a surfactant to a spiropyran.

a) Concentration

In the methods disclosed herein, the reaction with the spiropyrans represented by Formulae I-III and one or more hydrogels or hydrogel precursors can take place under various conditions. For example, the reaction can take place neat. In another aspect, the reaction can take place in any solvent. For example, the reaction can take place in an aqueous solvent, such as, but not limited to, water, aqueous hexane, aqueous ethanol, aqueous methanol, aqueous propanol, and the like. The reaction can also take place in non-aqueous solvents, such as, but not limited to, butanol, DMSO, DMF, THF, pyran, benzene, toluene, hexane, cyclohexane, pentane, cyclopentane, dichloromethane, dichloroethane, chloroform, carbon tetrachloride, tri and tetrachlorethane, octane, nitromethane, acetone, MEK, diethylether, diisopropyl ether, ethyl acetate, pyridine, and the like. In another example, the reaction can take place in a diphasic system containing an aqueous phase and an organic phase, such as those described herein. The amount of solvent used and the concentration of the spiropyran of Formulae I-III and/or hydrogel or hydrogel precursor will depend on the particular compound being prepared, the type of solvent, preference, and the like.

b) Temperature

The spiropyran of Formulae I-III can be reacted with the hydrogel or hydrogel precursor at any temperature sufficient to form a bond between the hydrogel precursor and the spiropyran. Typically, the reaction can take place at an elevated temperature. The precise elevated temperature can depend on the particular compounds being used, the solvent, the amount or concentration of the reagents, preference, and the like. Suitable temperatures at which the compositions disclosed herein can be reacted include, but are not limited to, from about 20 to about 200° C., from about 50 to about 220° C., from about 70 to about 240° C., from about 90 to about 260° C., or from about 110 to about 280° C. In other examples, the temperature of the reaction can be at about 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 300° C., where any of the stated values can form an upper or lower endpoint when appropriate.

B. Methods of making hydrogel

The disclosed compositions can be used to form hydrogels. Formation of hydrogels from the disclosed compositions can be accomplished as described by Dinsmore: Dinsmore, et al., Science 298:1006 (2002); Dinsmore, et al., Curr. Op. Colloid Interface Sci. 5:5-11 (1998); Dinsmore, et al., Appl. Opt. 40:4152 (2001); Dinsmore, et al., Nature 383:239 (1996); Lin, et al., Science 299:226 (2003); Lin, et al., J. Amer. Chem. Soc. 125:12690 (2003); Boker, et al., Nat. Mater. 3:302-306 (2004); Nikolaides, et al., Nature 420:299-301 (2002); Nikolaides, et al., Nature 424:1014 (2003); Dinsmore, et al., Appl. Phys. Lett. 75:802 (1999); Breen, Langmuir 17:903-907 (2001), which are each incorporated by reference herein for their teachings of methods for preparing hydrogels and colloidosomes from micro and nanoparticles.

Further, microgels and nanogels can be prepared and characterized from the disclosed compositions as described in Hu, et al., Science 269:525-527 (1995); Hu, et al., Nature 393:149-152 (1998); Hu, et al., Advanced Materials 12:1173-1176 (2000); Hu, et al., Adv. Mater. 13:1708 and cover (2001); Wu, et al., Physical Review Letters 90 (2003), which are incorporated by reference herein for their teaching of preparing micro and nanogels.

In one aspect, the disclosed compositions can be prepared by polymerizing a hydrogel precursor with a spiropyran. In another aspect, the hydrogel can be polymerized with a spiropyran in the absence of a surfactant. Also disclosed are compositions prepared by the disclosed methods. As noted, the disclosed compositions can be a microgel, nanogel, colloidosome. Further, the disclosed compositions can be decreased in size upon exposure to UV light or dark. In another example, the disclosed compositions can be increased in size upon exposure to visible light.

C. Pharmaceutical Formulation

Also, disclosed herein are pharmaceutical formulations. In one aspect, a pharmaceutical formulation can comprise any of the compositions disclosed herein with a pharmaceutically acceptable carrier. For example, a pharmaceutical formulation can comprise a hydrogel composition disclosed herein, an encapsulated or sequestered pharmaceutical active, and a pharmaceutically acceptable carrier. The disclosed pharmaceutical formulations can be used therapeutically or prophylactically.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (19th ed.) Gennaro, ed., Mack Publishing Company, Easton, Pa., 1995, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, antiinflammatory agents, anesthetics, and the like.

The pharmaceutical formulation can be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compounds can be administered orally, intravenously, intraperitoneally, intramuscularly, subcutaneously, intracavity, or transdermally.

Pharmaceutical formulations for oral administration include, but are not limited to, powders or granules, suspensions or solutions in water or non-aqueous media, capsules, sachets, or tablets. Thickeners, flavorings, diluents, emulsifiers, dispersing aids, anti-oxidants, or binders may be desirable.

Pharmaceutical formulations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Some of the formulations can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Examples of pharmaceutical actives that can be used in the disclosed hydrogels include, but are not limited to, adrenocortical steroid; adrenocortical suppressant; aldosterone antagonist; amino acid; anabolic; androgen; antagonist; anthelmintic; anti-acne agent; anti-adrenergic; anti-allergic; anti-amebic; anti-androgen; anti-anemic; anti-anginal; anti-arthritic; anti-asthmatic; anti-atherosclerotic; antibacterial; anticholelithic; anticholelithogenic; anticholinergic; anticoagulant; anticoccidal; antidiabetic; antidiarrheal; antidiuretic; antidote; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimitotic; antimycotic, antineoplastic, antineutropenic, antiparasitic; antiperistaltic, antipneumocystic; antiproliferative; antiprostatic hypertrophy; antiprotozoal; antipruritic; antipsoriatic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; diagnostic aid; diuretic; ectoparasiticide; enzyme inhibitor; estrogen; fibrinolytic; free oxygen radical scavenger; glucocorticoid; gonad-stimulating principle; hair growth stimulant; hemostatic; hormone; hypocholesterolemic; hypoglycemic; hypolipidemic; hypotensive; immunizing agent; immunomodulator; immunoregulator; immunostimulant; immunosuppressant; impotence therapy adjunct; inhibitor; keratolytic; LHRH agonist; liver disorder treatment, luteolysin; mucolytic; mydriatic; nasal decongestant; neuromuscular blocking agent; non-hormonal sterol derivative; oxytocic; plasminogen activator; platelet activating factor antagonist; platelet aggregation inhibitor; potentiator; progestin; prostaglandin; prostate growth inhibitor; prothyrotropin; pulmonary surface; radioactive agent; regulator; relaxant; repartitioning agent; scabicide; sclerosing agent; selective adenosine A1 antagonist; steroid; suppressant; symptomatic multiple sclerosis; synergist; thyroid hormone; thyroid inhibitor; thyromimetic; amyotrophic lateral sclerosis agents; Paget's disease agents; unstable angina agents; uricosuric; vasoconstrictor; vasodilator; vulnerary; wound healing agent; and/or xanthine oxidase inhibitor.

D. Methods of Use

The disclosed compositions have many uses. For example, the disclosed hydrogels can be applied to controlled drug release applications (Huang, et al., J. of Controlled Release 94:303-311(2004)). For example, disclosed herein is an approach to encapsulating materials (e.g., cells, small molecules, drugs, pharmaceuticals, and nutraceuticals) in self-assembled capsules with controlled architecture and permeability that can be dynamically changed (by exposure to light for example).

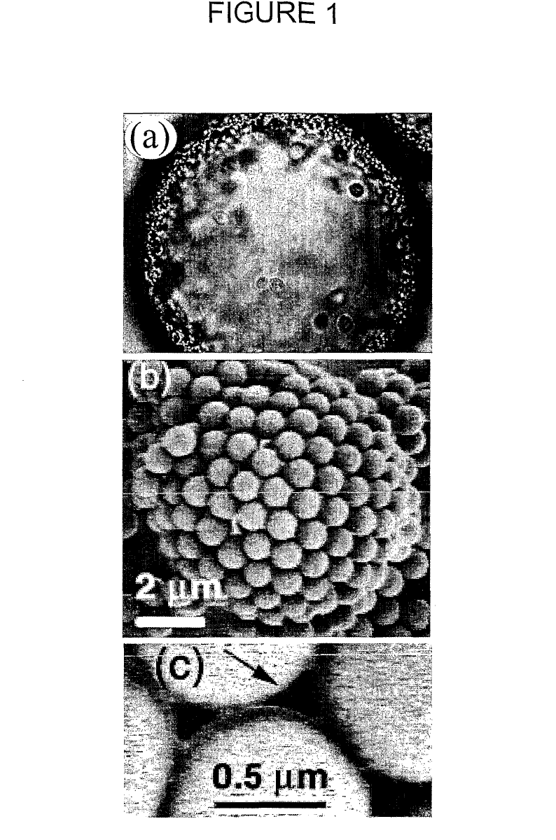

In a specific aspect, the compositions disclosed herein can be in a form known as colloidosomes (Dinsmore, et al., Science 298:1006 (2002); Gordon, et al., J. Am. Chem. Soc. 126:14117-14122 (2004)), which comprise spherical shells composed of a single, densely-packed layer of crosslinked nano- or microparticles (see FIG. 1). Interstices between the particles in the shell provide pores of precisely controlled size, allowing solvent and small molecules to permeate the shell. Larger encapsulated materials, however, are entrapped by the pores, which may be varied in size from a few nanometers to micrometers. Although colloidosomes have been reviewed with enthusiasm (Gouin, Trends Food Sci. Technol. 15:330-347 (2004); Dove, Nature Biotechnology 20:1213 (2002) to date the investigations have used inert model materials. By the methods disclosed herein, cells can be successfully encapsulated. The capsule can be formed by directed assembly.

Colloidosomes can be synthesized using monodisperse hydrogel nanoparticles as disclosed herein as building blocks. Also, as disclosed herein, photoactive spiropyrans can be incorporated into the hydrogels. In response to environmental stimuli such as temperature, pH, and light exposure, these hydrogel particles can change their volume by several orders of magnitude, changing the permeability of colloidosomes.

The disclosed composition (hydrogels, colloidosomes) can be used to deliver various agents and materials. The encapsulated material or loading material can include, but is not limited to, cells, bioactive yeast cells, pharmaceuticals, nutritional supplements, oligonucleotides (e.g., DNA), peptides, proteins, and the like. One specific use contemplated herein is the use of the disclosed compositions to deliver oligonucleotides such as DNA, RNA, and antisense oligonucleotides.

Antisense oligonucleotides (AS-ODN) show great potential as gene therapy agents, but are limited by the requirement for high doses, non-specific uptake, toxic side effects, and quick degradation. Moreover, due to their charge and polarity, they have low cellular uptake. To overcome these problems, various delivery vectors such as liposomes have been devised which have been able to achieve greater transfection efficiency. However, despite their ability to serve as depots for gene delivery, liposomes neither target specific tissues nor exhibit high levels of DNA release intracellularly. The future of gene therapy is in gene carriers that can target specific tissues to result in selective inhibition while avoiding any systemic toxicity (Fiset and Gounni, Rev. Biol. Biotechnol. 1:27 (2001)).

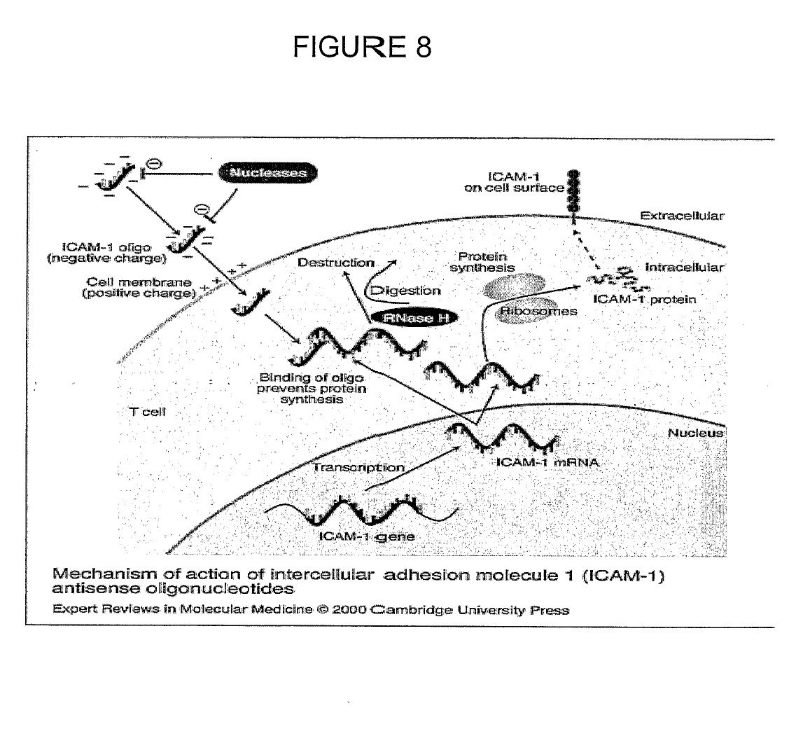

As an example, many approaches have been developed to suppress intracellular adhesion molecule-1 (ICAM-1) activity, which has an important role in inflammatory reactions, including anti-ICAM-1 antibodies, AS-ODNs for ICAM-1, and genetic knockouts (Kelly, et al., J. Clin. Inv. 97:1056 (1996); Haller, et al., Kidney Int. 53:1550 (1998). As shown in FIG. 8, AS-ODNs in particular have shown the greatest efficacy by improving functional kidney parameters such as serum creatinine levels and glomerular filtration rates as well as renal histology (Chen, et al., Transplantation 68:880 (1999); Dragun, et al., Kidney Int. 54:590 (1998)). Therefore, they have been implemented as preventative treatments in models of I/R injury, organ transplant rejection, and inflammatory diseases (Feeley, et al., Transplantation 69:1067 (2000); Chen, et al., Transplantation 68:880 (1997); Stepkowski, et al., Transplantation 66:699 (1998)). However, this promising AS-ODN treatment has been limited by the lack of effective delivery vectors and methods. Therefore, much of the current work in the field of gene therapy has been concerned with the discovery of safe, target-specific vectors for AS-ODN uptake into cells. These include viral vectors, cationic lipids and macromolecules, activated dendrimers, and polymeric nanoparticles (Kausch, et al., J. Urol. 168:239 (2002); Pouton, et al., Adv. Drug. Revs. 46:187 (2001); Ritter, et al., Curr. Gene Ther. 5:101 (2005); Dagon, et al., J. Gene Med. (2005); Shoji, et al., Curr. Pharm. Des. 7:785 (2004)). In particular, synthetic cationic polymer carriers such as lipofectin and DOTAP (N-(1-(2,3-dioleoxy)propyl)-N,N,N,-trimethylammonium methyl-sulfate) have shown promising results by forming a cationic complex with the anionic DNA that could electrostatically interact with the cell membrane, resulting in highly efficient endocytocis of the complex (Brown, et al., Int. J. Pharm. 229:1 (2001); Gao, et al., Gene Ther. 2:710 (1995)). Once internalized, the complex undergoes disassociation, releasing the DNA to inhibit its target genes.

A major drawback of this approach, however, was that the strong association bonds required between the polymer and DNA made it difficult for subsequent disassociation to take place inside the cell, resulting in low transfection efficiency (Weyermann, et al., J. Control Release, 100:411 (2004)). Many other conventional cationic polymer gene carriers have also been limited by the intermediate binding strengths of their complexes during gene transfection (Zhang, et al., J. Control Release 100:165 (2004)).

At the in vivo level, such gene therapies are further limited by the available methods employed to deliver these gene vectors to targeted tissues and organs. One technique that has been used is gene transfer into explanted cells followed by their implantation back into the appropriate tissues (Miller, Nature 357:455 (1992)). Other methods include intra-arterial injections, injection directly into the tissues, topical application, or uptake via inhalation (Galanis, et al., Crit. Rev. Oncol. Hematol. 38:177 (2001); Nakamura, et al., Gene Ther. 5:1455 (1998); Ledley, et al., Hum. Gene Ther. 6:1129 (1995)). These routes of administration have several drawbacks such as the transduction rarely being selective enough to use small quantities of the gene-containing vector, and the vector uptake not being sufficiently confined to the target organs leading to unwanted systemic prevalence of the genetic material and the associated side effects (Fiset, et al., Rev. Biol. Biotechnol. 1:27 (2001); Takakura, et al., Adv. Drug Deliv. Rev. 34:93 (1998); Bally, et al., Adv. Drug. Deliv. Rev. 38:291 (1999)). Moreover, even though over 14 antisense drugs were being tested in clinical trials in 2002, leading to the FDA approval of the first antisense drug for the treatment of human cytomegalovirus retinitis in patients with AIDS (Marwick, J. Amer. Med. Assoc. 280:871 (1998)), gene therapy trials have been impeded due to immunogenic and pathogenic problems associated with the vectors and delivery methods (Thomas, et al., Nature Genetics 4:346 (2003).

Acute renal failure is usually the result of diabetic nephropathy, hypertension, glomerulonephritis, and ischemic injury. Gene therapy has been attempted in experimental animals that have been modeled for each of these conditions, especially in the study of ischemic injury. In particular, the genes iNOS and ICAM-1 have been identified as important mediators of ischemic injury that can be targeted by gene therapy. Some of the vectors that have been used include liposomes, polycations, viral fusion proteins, electroporation, and hydrodynamic-based gene transfer. However, these techniques have experienced major challenges, including how to prolong and control transgene expression or antisense inhibition and how to minimize the adverse, non-specific side-effects of viral and nonviral vectors.

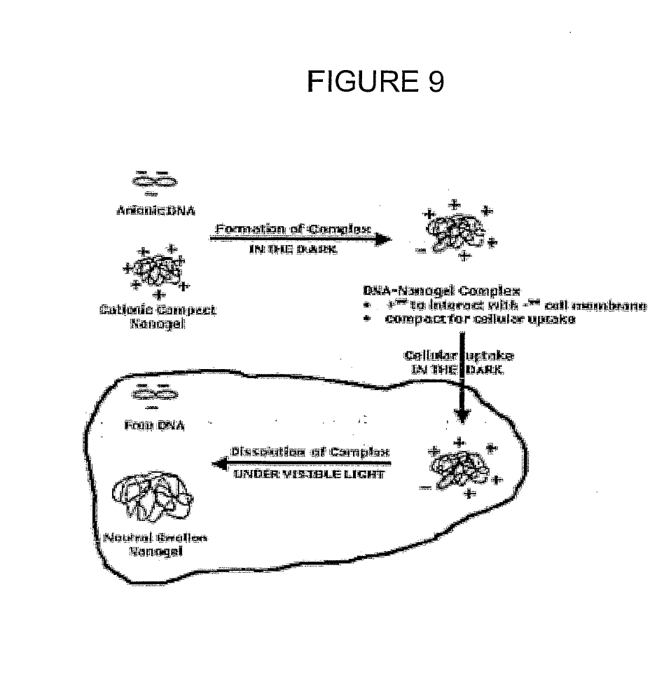

The hydrogels and methods disclosed herein address in vitro and in vivo limitations of gene therapy by using small quantities of nanogel polymers (e.g., PNIPAAm nanogel polymers) as vectors to deliver therapeutic AS-ODN to organs and tissues. PNIPAAm polymers have been identified as gene uptake vectors for DNA both in vitro and in vivo, proving to be capable of equally associating and disassociating strong complexes with DNA (Yokoyama, Drug Disc. Today 7:426 (2002); Hinrichs, et al., J. Cont. Release 60:249 (1999); Saunders and Vincent, Adv. Coll. Interface Sci. 80:1 (1999); Kurisawa, et al., J. Controlled Release 69:127 (2000)). Since the complex binding strength is not mutually exclusive, the compositions disclosed herein are an improvement over existing polymers which have not been efficient in selectively disassociating DNA during transfection. The disclosed compositions can have photoactive properties that can be bioengineered for various applications. When coupled with a spiropyran functional group, the composition contracts and expands upon exposure to light stimuli (Garcia, et al., J. Phys. Chem. 104:6103 (2000)). As a result, PNIPAAM can be selectively induced to associate or disassociate its contents by the use of an externally modulated light source. This makes it a highly-specific gene delivery vector that can be custom-tailored for a variety of biological systems (FIG. 9). Moreover, the strong complex formed between PNIPAAm and DNA has the effect of protecting the DNA from degradation by nucleases (Murata, et al., Bioorg. Med. Chem. Lett. 17:3967 (2003)), and PNIPAAm in a globule state has exhibited excellent cellular membrane permeability (Oupicky, et al., J. Controlled Release 65:149 (2000)). Another significant feature of the disclosed compositions as gene carriers is their low cytotoxicity which can be reduced even further as the charge density is decreased (Choksakulnimitr, et al., J. Controlled Release 34:233 (1995)). Therefore, by creating a photoactive nanogel polymer that can selectively and safely release active molecules such as DNA into cells, the transfection efficiency and therapeutic effects of AS-ODN for example can be enhanced.

Using the disclosed compositions to deliver nucleic acids, such as AS-ODN, can have a positive impact on the short life expectancies of the large numbers of ARF patients on dialysis or awaiting transplants. Blocking the destructive effects of ICAM-1 can improve healing and preserve organ function following I/R injury during cancer surgery, transplantation, or shock, while enhancing the effects of PAX-2 by inhibiting Activin-A could serve to regenerate injured kidney tissue. Moreover, the unique ability of the disclosed compositions to be externally modulated before and after incorporation offers a substantial benefit to patients by being more specific and effective while having less toxicity than systemic therapy.

The disclosed photoactive carrier technology can also be applied in a variety of medical specialties to help overcome other sources of ischemic injury seen during vascular surgery, heart and lung transplants, and after cerebrovascular accidents. It can also be used to target a wide range of diseases including, for example, cancer, cystic fibrosis, alpha-1-anti-tripsine deficiency, and familial colon polyposis, where efficient transfection of genes is a major challenge. As a result, this delivery system can be used as a universal vector for many different purposes with the ability to be custom-tailored for specific organ systems.

In another example, the disclosed hydrogels can be coupled to a targeting moiety and targeted to a particular cell type, via antibodies, receptors, or receptor ligands. The following references are examples of the use of this technology to target specific tissue (Senter, et al., Bioconjugate Chem 2:447-51, 1991; Bagshawe, Br J Cancer 60:275-81, 1989; Bagshawe, et al., Br J Cancer 58:700-3, 1988; Senter, et al., Bioconjugate Chem 4:3-9, 1993; Battelli, et al., Cancer Immunol Inmunother 35:421-5, 1992; Pietersz and McKenzie, Immunolog Reviews 129:57-80, 1992; and Roffler, et al., Biochem Pharmacol 42:2062-5, 1991). These techniques can be used for a variety of other specific cell types.

1. Dosage

When used in the above described methods or other treatments disclosed herein, an “effective amount” of one of the disclosed compounds can be employed in pure form or, where such forms exist, in pharmaceutically acceptable salt form and with or without a pharmaceutically acceptable excipient, carrier, or other additive.

The specific effective dose level for any particular subject will depend upon a variety of factors including the condition or disease being treated and the severity of the condition or disease; activity of the specific compound employed; the specific composition employed; the age, body weight, general health, sex and diet of the subject; the time of administration; the route of administration; the rate of excretion of the specific compound employed; the duration of the treatment; drugs used in combination or coincidential with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose.

The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products.

2. Administration and Delivery

In one aspect, disclosed herein are uses of a hydrogel composition to deliver a loading substance to a subject, wherein the microcapsule contains any of the compounds disclosed herein. Also disclosed are methods for delivering a compound (e.g., DNA) to a subject by administering to the subject any of the compositions disclosed herein.

The compositions disclosed herein can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

### VI. EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, desired solvents, solvent mixtures, temperatures, pressures and other reaction ranges and conditions that can be used to optimize the methods described herein. Only reasonable and routine experimentation will be required to optimize such process conditions.

In the following examples, N-isopropylacrylamide was obtained from Polysciences Inc. (Warrington, Pa.) and used as received. The cross-linker N,N′-methylenebis(acrylamide) (MBAAM) was purchased from Bio-Rad Co. (Hercules, Calif.). The potassium persulfate (KPS) were both bought from Aldrich Chemical Co. (Milwaukee, Wis.) and used as received. Distilled and deionized water (resistance of 18 Mø,cm) was used throughout. A 0.5 μm Millipore (Millex LCR25; Millipore, Billerica, Miss.) filter was used to purify the dilute sample solutions.

### A. EXAMPLE 1

### Synthesis of PNIPAM-SP Nanoparticles

PNIPAAm-SP nanoparticles were prepared by copolymerizing NIPAAM along with a spiropyran derivative having an unsaturated allylamide linker (see FIG. 2 bottom panel); this is a modification of the precipitation polymerization method of Pelton and Chibante, Coll. Surf 20:247 (1986). Spiropyran allylamide (0.009 g) was dissolved in pH 9, NaOH/deionized water solution at 40 to 50° C. Then, NIPAAm monomer (0.6 g) and a cross link agent N,N′-methylene-bis-acrylamide (BIS) (0.013 g) were added into this solution. The solution was stirred at 300 rpm for 30 min under a nitrogen environment. An initiator, potassium persulfate (KPS) (0.02 g), dissolved in 2 mL of deionized water was added to start the reaction. Two batches of pre-gel solutions were prepared with pH 6 and pH 8, respectively. The reaction was taken at temperature at 70° C. under N₂ gas for 4 hours under dark to ensure that all the monomer was reacted. Raising the temperature to 70° C. was needed to have precipitation polymerization. After cooling to room temperature, the PNIPAAm-SP nanoparticle dispersions were condensed using an ultracentrifuge with speed of 40,000 rpm for 1 hour (h).

Based on the mole % of spiropyran incorporated within the reaction media, the hydrogels were estimated to contain about 1% of spiropyran. Epifluorescence microscopy of the macrogels and fluorescence spectroscopy of the nanogels showed that they fluoresced when they were irradiated with UV light or after being left in the dark, and were non-fluorescent after visible irradiation. This indicated that the pendant spiropyran groups in the polymer were still photochemically active after the polymerization.

### B. EXAMPLE 3

### Composition Characterization

The nanoparticles prepared according to Example 1 where characterized by light scattering measurements. For these measurements, 10 mL aliquot samples were taken from the reaction container at different times after the reaction started; all aliquots were dialyzed for dynamic light scattering analysis. A commercial laser light scattering spectrometer (ALV, Co., Germany) was used with a helium-neon laser (Uniphase 1145P, output power of 22 mW and wavelength of 632.8 nm) as the light source.

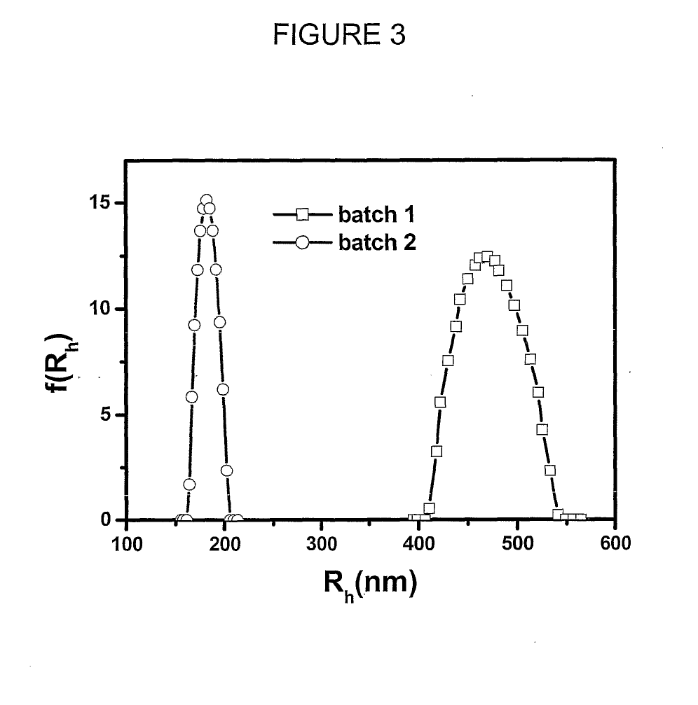

FIG. 3 shows the hydrodynamic radius distributions (ƒ(Rₕ)) of PNIPAAM-SP nanoparticles prepared according to Example 1 under dark in deionized water at pH 6 and pH 8 respectively. At pH 6, PNIPAAm-SP nanoparticles have an average hydrodynamic radius around 500 nm, similar to ones obtained by Pelton without surfactant (Pelton and Chibante, Coll. Surf. 20:247 (1986)). However, at pH 8, the resultant PNIPAAm-SP nanoparticles have Rₕ around 150 nm. Such small PNIPAAm nanoparticles were usually made in the presence of surfactant. While not wishing to be bound by theory, it is believed that this may be because SP became ionized in dark at pH 8 than pH 6 so that each molecule acts like a surfactant molecule. In practice, complete removal of a surfactant from the resulted hydrogel precursors can be desired for biomedical applications. This study revealed that spiropyran SP at pH 8 acted as a surfactant, resulting in surfactant-free monodisperse PNIPAM-SP nanoparticles with particle size smaller than 300 nm. After polymerization, spiropyran was covalently bonded into the PNIPAAm network and changed its role from a surfactant to a spiropyran.

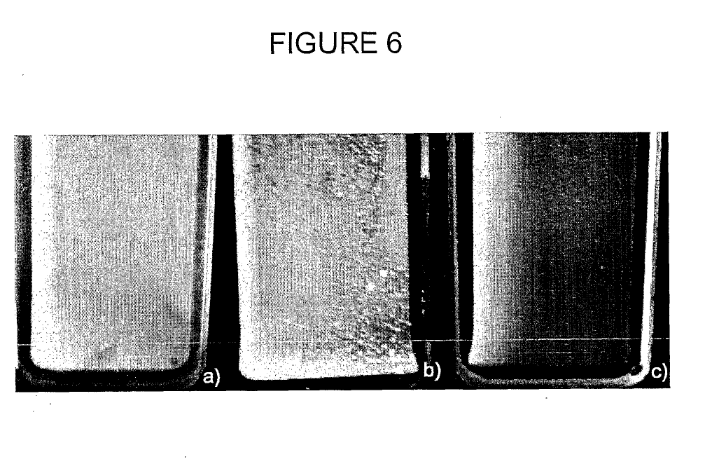

Further, these nanoparticles of Example 1 can change their size under various light conditions. Specifically, when exposed to visible light, the spiropyran undergoes an isomerization wherein the spiro linkage is severed, resulting in a highly polar “open” form that is colored (typically absorbing near 530 nm). This causes the particles to expand as shown in FIG. 6a. The volume phase transition in gels can be due to the change in the osmotic pressure by the external stimuli, and the rate-determining step of the deformation is the diffusion process. Therefore, the response can be too slow for bulk gels. Due to small dimension, the responsive rate of PNIPAM-SP nanoparticles can be much faster.

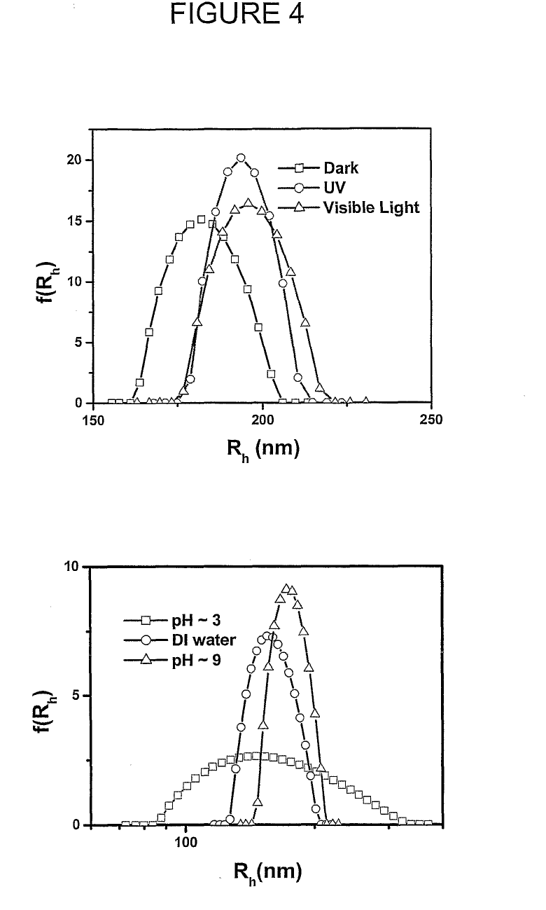

These nanoparticles also change their size in response to pH changes as shown in FIG. 4 bottom graph. At higher pH (e.g., pH 9), the particles expand due to the polar “open” form. One reason to study spiropyran-NIPA hydrogels is that unlike azobenzene and leucohydroxides, upon UV irradiation spiropyran is converted to a zwitterionic form in aqueous solution. This leads to more versatile charge-electric field applications at different pH values along with unique interactions with biological molecules.

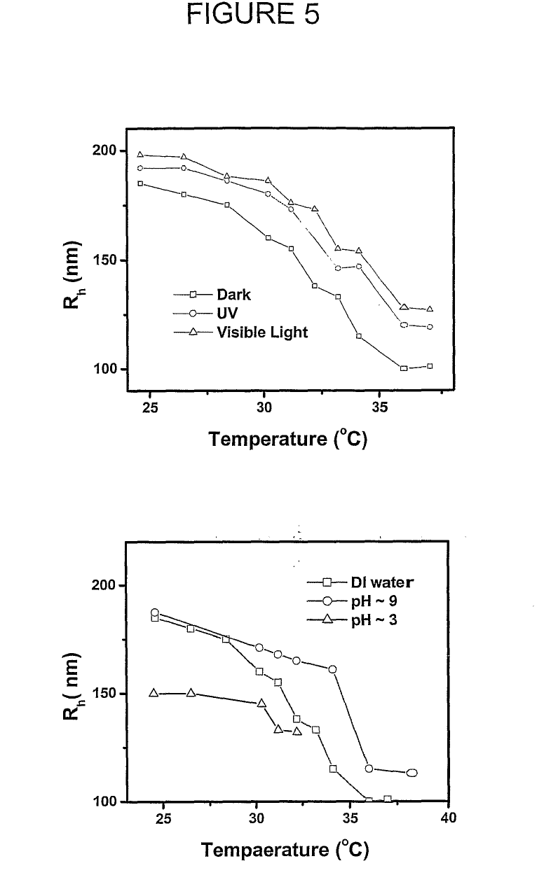

The hydrodynamic radius of PNIPAAM-SP nanoparticles is plotted as a function of temperature at various light conditions (FIG. 5 top graph) and at various pH values (FIG. 5 bottom graph). Under all temperatures studied (15° to 35° C.), the UV irradiated particles were always smaller than those under visible irradiation. Electrophoresis measurements performed on the nanogels confirmed that the particles underwent a large increase in surface charge (from 0.001 to 0.020 C/m²) when the wavelengths of irradiation were changed from visible to UV. In contrast, macroscopic samples (“macrogels”) were found to swell about 10% under UV irradiation relative to under visible irradiation. While not wishing to be bound by theory, it is believed that this can be explained by the UV-induced ionic groups on the spiropyrans. It is further believed that in the nanogels, the UV-generated polar groups undergo aggregation due to static electric interaction that causes the particles to shrink.

The temperature that the radius versus temperature curves undergoes the sharpest change is defined as the volume phase transition temperature T_{c}. Like the neutral PNIPAmM gel, the PNIPAAm-SP nanoparticles undergo a drastic volume change from a swollen state for T less than T_{c} to a collapsed state for T greater than T_{c}, where T_{c} is approximately 34° C. The value of T_{c} can be increased by copolymerization of polar molecules or decreased by copolymerization of nonpolar molecules. In the compositions disclosed herein, T_{c} is smaller for PNIPAAm-SP nanoparticles under dark than under visible light (FIG. 5 top graph), indicating that the attractive interaction force between charged SP ions dominate. On the other hand, T_{c} is smaller at pH 3 than at pH 9 (FIG. 5 bottom graph), indicating that the gels are more hydrophobic in lower pH. While not wishing to be bound by theory, it is believed that there are attractive interactions among SP molecules at lower pH.

Under dark and pH 8, the SP molecule behaves like a surfactant. Without any surfactant, a particle radius of approximately 150 nm can be obtained. This confirms the surfactant-like effect of the SP. Such small sized PNIPAAm nanoparticles can only be obtained in the presence of a surfactant. More remarkably, the surfactant free PNIPAM-SP particles were also monodisperse and can self-assemble into a crystalline lattice at polymer concentration around 8 weight %. The PNIPAAM-SP nanoparticles have been concentrated using ultra-centrifugation with the speed of 40,000 rpm for 2 h. Aqueous dispersions of these particles with polymer concentration around 8 weight % exhibit bright colors, indicating the formation of an ordered structure as shown in FIG. 6a. This structure has been further confirmed by measuring the turbidity of the samples using a UV-visible spectroscopy (Agilent 8453). Corresponding to the appearance of colors, the turbidity of the dispersions exhibits a sharp peak at a certain wavelength λ_{c} as shown in FIG. 6b. The color originates from Bragg diffraction. Constructive interference occurs if Bragg condition 2nd sin θ=mλ, is satisfied, where d, θ, n, λ, m are the lattice spacing, the diffraction angle, the refractive index of the gel medium, the wavelength of light in vacuum and an integer, respectively. The peak disappears when the temperature is above T_{c}.

In summary, in additional to thermally responsive behavior that is associated with the PNIPAM gel, the nanoparticles disclosed herein change their volume and hydrophobicity in response to light and pH. Studies of the swelling of PNIMAAm-SP particles revealed the astonishing result that the nanoscale-sized particles shrink upon UV irradiation (FIG. 3), whereas the macro-gels swell. Under all temperatures studied (15°-35° C.), the UV irradiated particles were always smaller than those under visible irradiation. The most dramatic difference occurs at 33° C. where the VIS irradiated particles are 520 nm in radius while the UV irradiated particles are at 260 nm radius (results for this sample not shown). Electrophoresis measurements performed on the nanogels confirmed that the particles underwent a large increase in surface charge (from about 0.001 to about 0.020 C/m²) when the wavelengths of irradiation were changed from visible to UV. In contrast, macroscopic samples (macrogels) were found to swell about 10% under UV irradiation relative to under visible irradiation, which can be explained by the UV-induced ionic groups on the spiropyrans. We theorize that in the nanogels, the UV-generated polar groups undergo aggregation that causes the particles to shrink.

### C. EXAMPLE 3

Proposed Modifications

To provide functional groups to the particles of Example 1, at least three different chemicals can be added to the pre-polymer solution, respectively: acrylic acid (AA), 2-hydroxyethyl acrylate (HEAc), and allylamine. The AA, HEAc, and allylamine provide carboxyl (—COOH), hydroxyl (—OH), and amine (NH₃) groups, respectively, which can serve as crosslinking sites to neighboring particles. Various schemes have been proposed to bond nanoparticles with different functional groups (Z. B. Hu, X. Lu, J. Gao, “Hydrogel Opals,” Adv. Mater. 13, 1708 and cover (2001); Hu and Huang, Angew. Chemie, Int. Ed. 42:4799 (2003)).

### D. EXAMPLE 4

Monodisperse nanoparticles of poly-N-isopropylacrylamide (PNIPAAm) were synthesized and were able to be controlled by their lower critical solution temperature (LCST). The LCST was easily modulated by the addition of hydrophilic co- monomers to the polymer chain, leading to the custom synthesis of these molecules possible. During controlled release experiments using DNA and dextran markers, the nanogel showed optimum release at temperatures below the LCST, as it expanded and disassociated the DNA. Moreover, it was found that the binding strength during association (loading) and subsequent disassociation (delivery) of these complexes was not mutually inclusive. Therefore, the composition was able to protect its contents from degradation while equally retaining the ability to efficiently release its contents. As a result, this composition can offer substantial improvements over conventional cationic gene carriers which require high doses to exert an effect because of their low binding strengths (Huang, et al., J. Controlled Release 94: 303 (2004)). t,1-23/2

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### VII. REFERENCES

R. G. Willaert, G. V. Baron, “Gel Entrapment and Micro-Encapsulation: Methods, Applications and Engineering Principles,” Rev. Chem. Eng. 12:5-205 (1996).

R. P. Lanza, R. Langer, J. Vacanti, Principles of Tissue Engineering (Academic Press, San Diego, 2000).

E. L. Chaikof, “Engineering and Material Considerations in Tissue Cell Transplantation,” Annu. Rev. Biomed. Eng. 1:103-127 (1999).

T. Bodeutsch, E. A. James, J. M. Lee, “The Effect of Immobilization on Recombinant Protein Production in Plant Cell Culture,” Plant Cell Reports 20:562-566 (2001).

C. Decamps, S. Norton, D. Poncelet, R. J. Neufeld, “Continuous Pilot Plant-Scale Immobilization of Yeast in Kappa-Carrageenan Gel Beads,” Aiche Journal 50:1599-1605 (2004).

G. Bergers, D. Hanahan, “Cell Factories for Fighting Cancer,” Nature Biotech 19:20-21 (2001).

T. A. Desai, D. J. Hansford, M. Ferrari, “Micromachined Interfaces: New Approaches in Cell Immunoisolation and Biomolecular Separation,” Biomol. Eng. 17:23-26 (2000).

J. K. Park, H. N. Chang, “Microencapsulation of Microbial Cells,” Biotechnol. Adv. 18:303-319 (2000).

G. Orive, R. M. Hernandez, A. R. Gascon, R. Calafiore, T. M. S. Chang, P. d. Vos, G. Hortelano, D. Hunkeler, I. Lacik, J. L. Pedraz, “History, Challenges and Perspectives of Cell Microencapsulation,” Trends Biotechnol. 22:87 (2004).

K. D. Green, I. S. Gill, J. A. Khan, E. N. Vulfson, “Microencapsulation of Yeast Cells and Their Use as a Biocatalyst in Organic Solvents,” Biotech. Bioeng. 49:535-543 (1996).

R. Gref, Y. Minamitake, M. T. Peracchia, V. Trubetskoy, V. Torchilin, R. Langer, “Biodegradable Long-Circulating Polymeric Nanospheres,” Science 263: 1600 (1994).

J. K. Mills, D. Needham, “Targeted Drug Delivery,” Expert Opin. Ther. Patents 9:1499-1513 (1999).

S. Gouin, “Microencapsulation: Industrial Appraisal of Existing Technologies and Trends,” Trends Food Sci. Technol. 15:330-347 (2004).

M. I. Re, “Microencapsulation by Spray Drying,” Drying Technology 16:1195-1236 (1998).

J. D. Dziezak, “Microencapsulation and Encapsulated Ingredients,” Food Technology—Chicago 42:136-151 (1988).

B. F. Gibbs, S. Kermasha, I. Alli, C. N. Mulligan, “Encapsulation in the Food Industry: A Review,” Int. J Food Sci. Nutr. 50:213-224 (1999).

A. D. Dinsmore, M. F. Hsu, M. G. Nikolaides, M. Marquez, A. R. Bausch, D. A. Weitz, “Colloidosomes: Self-Assembled, Selectively-Permeable Capsules Composed of Colloidal Particles,” Science 298:1006 (2002).

V. D. Gordon, C. Xi, J. W. Hutchinson, A. R. Bausch, M. Marquez, D. A. Weitz, “Self-Assembled Polymer Membrane Capsules Inflated by Osmotic Pressure,” J. Am. Chem. Soc. 126:14117-14122 (2004).

A. Dove, “Research News: Designer Coatings,” Nature Biotech. 20:1213 (2002).

N. A. Peppas, Hydrogels in Medicine and Pharmacy (CRC Press, Boca Raton, Fla., 1987).

N. A. Peppas, R. Langer, “New Challenges in Biomaterials,” Science 263:1715 (1994).

A. D. Dinsmore, J. C. Crocker, A. G. Yodh, “Self-Assembly of Colloidal Crystals,” Curr. Op. Colloid Interface Sci. 5:5-11 (1998).

A. D. Dinsmore, E. R. Weeks, V. Prasad, A. C. Levitt, D. A. Weitz, “Three-Dimensional Confocal Microscopy of Colloids,” Appl. Opt. 40: 4152 (2001).

A. D. Dinsmore, A. G. Yodh, D. J. Pine, “Entropic Control of Particle Motion Using Passive Surface Microstructures,” Nature 383:239 (1996).

Y. Lin, H. Skaff, T. S. Emrick, A. D. Dinsmore, T. P. Russell, “Nanoparticle Assembly and Transport and Liquid-Liquid Interfaces,” Science 299:226 (2003).

Y. Lin, H. Skaff, A. Boker, A. D. Dinsmore, T. Emrick, T. P. Russell, “Ultrathin Crosslinked Nanoparticle Membranes,’ J. Amer. Chem. Soc. 125:12690 (2003).

A. Boker, Y. Lin, K. Chiapperini, R. Horowitz, M. Thompson, V. Carreon, T. Xu, C. Abetz, H. Skaff, A. D. Dinsmore, T. Emrick, T. P. Russell, “Hierarchical Nanoparticle Assemblies Formed by Decorating Breath Figures,” Nat. Mater. 3:302-306 (2004).

M. G. Nikolaides, A. R. Bausch, M. F. Hsu, A. D. Dinsmore, M. P. Brenner, C. Gay, D. A. Weitz, “Electric-Field-Induced Capillary Attractions between Like-Charged Particles at Liquid Interfaces,” Nature 420:299-301 (2002).

M. G. Nikolaides, A. R. Bausch, M. F. Hsu, A. D. Dinsmore, M. P. Brenner, C. Gay, D. A. Weitz, “Electric-Field-Induced Capillary Attractions between Like-Charged Particles at Liquid Interfaces (Reply),” Nature 424:1014 (2003).

A. D. Dinsmore, D. S. Hsu, H. F. Gray, S. B. Qadri, Y. Tian, B. R. Ratna, “Mn-Doped Nanoparticles as Efficient Low-Voltage Cathodoluminescent Phosphors,” Appl. Phys. Lett. 75:802 (1999).

M. L. Breen, A. D. Dinsmore, R. H. Pink, S. B. Qadri, B. R. Ratna, “Sonochemically Produced Zns-Coated Polystyrene Core-Shell Particles for Use in Photonic Crystals,” Langmuir 17:903-907 (2001).

P. B. Umbanhowar, V. Prasad, D. A. Weitz, “Monodisperse Emulsion Generation Via Drop Break Off in a Coflowing Stream,” Langmuir 16:347-351 (2000).

P. Pieranski, “Two-Dimensional Interfacial Colloidal Crystals,” Phys. Rev. Lett. 45:569-572 (1980).

S. U. Pickering, “Emulsions,” J. Chem. Soc. 91:2001 (1907).

B. P. Binks, S. O. Lumsdon, “Pickering Emulsions Stabilized by Monodisperse Latex Particles: Effects of Particle Size,” Langmuir 17:4540-4547 (2001).

S. Tarimala, L. L. Dai, “Structure of Microparticles in Solid-Stabilized Emulsions,” Langmuir 20:3492-3494 (2004).

O. D. Velev, K. Nagayama, “Assembly of Latex Particles by Using Emulsion Droplets as Templates. 3. Reverse (Water in Oil) System,” Langmuir 13:1856-1859 (1997).

L. M. Croll, H. D. H. Stover, “Formation of Tectocapsules by Assembly and Cross-Linking of Poly(Divinylbenzene-Alt-Maleic Anhydride) Spheres Are the Oil-Water Interface,” Langmuir 19:5918 (2003).

P. F. Noble, O. J. Cayre, R. G. Alargova, O. D. Velev, V. N. Paunov, “Fabrication of “Hairy” Colloidosomes with Shells of Polymeric Microrods,” J. Am. Chem. Soc. 126:8092-8093 (2004).

H. Wang, E. K. Hobbie, “Amphiphobic Carbon Nanotubes as Macroemulsion Surfactants,” Langmuir 19:3091-3093 (2003).

T. Reincke, S. G. Hickey, W. K. Kegel, D. Vanmaekelbergh, “Spontaneous Assembly of a Monolayer of Charged Gold Nanocrystals at the Water/Oil Interface,” Angew. Chem.—Int. Edit. 43:458-462 (2004).

H. Duan, D. Wang, D. G. Kurth, H. Mohwald, “Directing Self-Assembly of Nanoparticles at Water/Oil Interfaces,” Ang. Chem. Int. Ed. 43:5639 (2004).

A. D. Dinsmore, D. A. Weitz, “Direct Imaging of Three-Dimensional Structure and Topology of Colloidal Gels,” J. Phys. Condens. Matt. 14:7581 (2002).

Y. Li, T. Tanaka, “Phase Transition of Gels,” Annu. Rev. Mat. Sci. 22:243 (1992).

A. S. Hoffman, “Hydrogels for Biomedical Applications,” Adv. Drug Delivery Rev. 54:3 (2002).

R. A. Siegel, B. A. Firestone, “Ph-Dependent Equilibrium Swelling Properties of Hydrophobic Poly-Electrolyte Copolymer Gels,” Macromolecules 21:3254-3259 (1988).

B. Jeong, Y. H. Bae, D. S. Lee, S. W. Kim, “Biodegradable Block Copolymers as Injectable Drug-Delivery Systems,” Nature 388:860-862 (1997).

C. Wang, R. J. Stewart, J. Kopecek, “Hybrid Hydrogels Assembled from Synthetic Polymers and Coiled-Coil Protein Domains,” Nature 397:417-420 (1999).

S. H. Gehrke, “Synthesis, Equilibrium Swelling, Kinetics Permeability and Applications of Environmentally Responsive Gels,” Adv. Polym. Sci. 110:81 (1993).

Y. Osada, H. Okuzaki, H. Hori, “A Polymer Gel with Electrically Driven Motility,” Nature 355:242-244 (1992).

Z. B. Hu, X. M. Zhang, Y. Li, “Synthesis and Application of Modulated Polymer Gels,” Science 269:525-527 (1995).

Z. B. Hu, Y. Y. Chen, C. J. Wang, Y. D. Zheng, Y. Li, “Polymer Gels with Engineered Environmentally Responsive Surface Patterns,” Nature 393:149-152 (1998).

Z. B. Hu, X. H. Lu, J. Gao, C. J. Wang, “Polymer Gel Nanoparticle Networks,” Adv. Mater. 12:1173-1176 (2000).

Z. B. Hu, X. Lu, J. Gao, “Hydrogel Opals,” Adv. Mater. 13:1708 and cover (2001).

J. Z. Wu, B. Zhou, Z. B. Hu, “Phase Behavior of Thermally Responsive Microgel Colloids,” Physical Rev. Lett. 90 (2003).

G. Huang, J. Gao, Z. B. Hu, J. V. S. John, B. C. Ponder, D. Moro, “Controlled Drug Release from Hydrogel Nanoparticle Networks,” J. Controlled Release 94:303-311 (2004).

A. A. Garcia, S. Cherian, J. Park, D. Gust, F. Jahnke, R. Rosario, “Photon-Controlled Phase Partitioning of Spiropyrans,” J. Phys. Chem. A 104:6103-6107 (2000).

R. Rosario, D. Gust, M. Hayes, F. Jahnke, J. Springer, A. A. Garcia, “Photon-Modulated Wettability Changes on Spiropyran-Coated Surfaces,” Langmuir 18:8062-8069 (2002).

R. Rosario, D. Gust, M. Hayes, J. Springer, A. A. Garcia, “Solvatochromic Study of the Microenvironment of Surface-Bound Spiropyrans,” Langmuir 19:8801-8806 (2003).

R. Rosario, A. A. Garcia, D. Gust, M. Hayes, J. Springer, in Proceedings of Spie: 4807. Physical Chemistry of Interfaces and Nanomaterials J. Zhang, Z. Wang, Eds. (2002) pp. 197.

R. H. Pelton, P. Chibante, “Preparation of Aqueous Latices with N-Isopropylacrylamide,” Coll. Surf. 20:247 (1986).

R. C. Bertelson, in Photochromism G. H. Brown, Ed. (Wiley-Interscience, New York, 1971).

B. C. Bunker, B. I. Kim, J. E. Houston, R. Rosario, A. A. Garcia, M. Hayes, D. Gust, S. T. Picraux, “Direct Observation of Photo Switching in Tethered Spiropyrans Using the Interfacial Force Microscope,” Nano Lett. 3:1723-1727 (2003).

S. Hirotsu, Y. Hirokawa, T. Tanaka, “Volume-Phase Transitions of Ionized N-Isopropylacrylamide Gels,” J. Chem. Phys. 87:1392-1395 C1987).

O. H. Kwon, A. Kikuchi, M. Yamato, Y. Sakurai, T. Okano, “Rapid Cell Sheet Detachment from Poly(N-Isopropylacrylamide)-Grafted Porous Cell Culture Membranes,” J. Biomed. Mater. Res. 50:82 (2000).

H. Weng, J. Zhou, L. P. Tang, Z. B. Hu, “Tissue Responses to Thermally-Responsive Hydrogel Nanoparticles,” J. Biomater. Sci., Polymer Ed. 15:1167 (2004).

Z. B. Hu, G. Huang, “A New Route to Crystalline Hydrogels as Guided by a Phase Diagram,” Angew. Chemie, Int. Ed. 42:4799 (2003).

X. H. Lu, Z. B. Hu, J. Gao, “Synthesis and Light Scattering Study of Hydroxypropyl Cellulose Microgel,” Macromolecules 33:8698 (2000).

K. Kyyronen, L. Hume, L. M. Benedetti, A. Urtti, E. Topp, V. J. Stella, “Methyleprednisolone Esters of Hyaluronic Acid in Ophthalmic Drug Delivery: In vitro and in vivo Release Studies,” Int. J. Pharm. 80:161 (1992).

K. C. Lowe, P. Anthony, M. R. Davey, J. B. Power, “Culture of Cells at Perfluorocarbon-Aqueous Interfaces,” Art. Cells, Blood Subs. Immob. Biotech. 27:255-261 (1999).

H. L. Holland, “Microbial Transformations,” Curr. Opin. Chem. Biol. 2:77-84 (1998).

Q. Jiang, S. J. Yao, L. H. Mei, “Tolerance of Immobilized Baker's Yeast in Organic Solvents,” Enzyme Microb. Technol. 30:721-725 (2002).

R. Leon, P. Fernandes, H. M. Pinheiro, J. M. S. Cabral, “Whole-Cell Biocatalysis in Organic Media,” Enzyme Microb. Technol. 23:483-500 (1998).

O. Rotthaus, M. Demuth, “Efficient Cyclization of Squalene Epoxide to Lanosterol with Immobilized Cells of Baker's Yeast,” Tetrahedron 58:7291-7293 (2002).

J. Qun, Y. Shanjing, M. Lehe, “Tolerance of Immobilized Baker's Yeast in Organic Solvents,” Enzyme Microb. Technol. 30:721 (2002).

Q. Yi, D. B. Sarney, J. A. Khan, E. N. Vulfson, “A Novel Approach to Biotransformations in Aqueous-Organic Two-Phase Systems: Enzymatic Synthesis of Alkyl Beta-D-Glucosides Using Microencapsulated Beta-Glucosidase,” Biotechnol. Bioeng. 60:385-390 (1998).

National Center for Health Statistics (NCHS), Centers for Disease Control and Prevention (CDC), U.S. Department of Health and Human Services (DHHS): Atlanta: Summary Health Statistics for U.S. Adults: National Health Interview Survey, 209, 2002.

National Institute of Diabetes and Digestive and Kidney Diseases, National Institutes of Health (NIH), U.S.R.D. “USRDS 2002 Annual Data Report.” DHHS, 2002.

S. H. Hou, D. A. Bushinsky, J. B. Wish, J. J. Q. Cohen, et al., “Hospital acquired renal insufficiency: a prospective study.” Am. J. Med. 74:243 (1983).

J. Kaufman, M. Dhakal, B. Patel, R. Hamburger, “Community-acquired acute renal failure.” Am J Kidney Dis, 17:191 (1991).

Shusterman, N., Strom, B. L., Murray, T. G., “Risk factors and outcome of hospital-acquired acute renal failure: clinical epidemiologic study.” Am. J. Med. 83:65 (1987).

Turney, J. H., Marshall D. H., Brownjohn, A. M., Ellis, “The evolution of acute renal failure, 1956-1988.” Q. J. Med. 74:83 (1990).

Wolfe, R., Ashby, V., et al., “Comparison of mortality in all patients on dialysis, patients on dialysis awaiting transplantation, and recipients of a first cadaveric transplant.” N. Engl. J. Med. 341:1725 (1999).

Liaño, F., Pascual, J, and the Madrid ARF Study Group, “Epidemiology of acute renal failure: A prospective, multicenter, community-based study.” Kidney Int, 50:811 (1996).

United Network for Organ Sharing: Organ Procurement and Transplantation Database (2003).

Horl, M. P., Schmitz, M., Ivens, K., Grabensee, B., “Opportunistic infections after renal transplantation.” Curr Opin Urol, 12:115 (2002).

Jindal, R. M., and Hariharan, S., “Chronic rejection in kidney transplants. An in-depth review.” Nephron. 83:13 (1999).

Arias, M., Escallada, R., De Francisco, A. M., Emilio, “Return to dialysis after renal transplantation. Which would be the best way?” Kidney Int. 61:85 (2002).

Lumsdaine, J. A., Wigmore, S. J., and Forsythe, J. L. R., “Recurrent glomerulonephritis following renal transplantation.” Transplantation 63:1045 (1997).

Brivet, F. G., Kleinknecht, D. J., Loirat, P., Landais, P. J., “Acute renal failure in intensive care units-causes, outcome and prognostic factors of hospital mortality: A prospective, multicenter study.” Crit Care Med 24:192 (1995).

Bonventre, J. V., “Mechanisms of ischemic acute renal failure.” Kidney International 43:1160 (1993).

Witzgall R., Brown D., Schwarz C., Bonventre J V., “Localization of proliferating cell nuclear antigen, vimentin, c-fos, and clusterin in the post-ischemic kidney.” J. Clin Invest, 93:2175 (1994).

Toback, F. G., “Regeneration after acute tubular necrosis.” Kidney Int. 41:226 (1992).

Mather J. P., Moore A., Li, R. H. “Activins inhibins and follistatins: Further thoughts on growing family of regulators.” Proc. Soc. Exp. Biol. Med. 215:209 (1997).

Ying S. Y., Zhang Z., Furst B., Batres Y., Huang G., Guangwu L., “Activins and activin receptors in cell growth.” Proc. Soc. Exp. Biol. Med. 214:114 (1997).

Maeshima A., Zhang Y. Q., Furukawa M., Naruse T., Kojima I.: Hepatocyte growth factor induces branching tubulogenesis by modulating the activin-follistatin system. Kidney Int, 58:1511, 2000.

Ritvos O., Tuuri T., Eramaa M., Sainio K., Hiden K., Saxen L., Gilbert S. F.: Activin disrupts epithelial branching morphogenesis in developing glandular organs of the mouse. Mech Dev, 50:229, 1995.

Maeshima A., Shiozaki S., Tajima T., Nakazato Y., Naruse T., Kojima I.: Number of glomeruli is increased in transgenic mice expressing truncated type II activin receptor. Biochem Biophys Res Commun, 268:445, 2000.

Lambert-Messerlian, G. M., Pinar, H., Laprade, E., Tantravahi, U., Schneyer, A., Canick, J. A. Inhibins and activins in human fetal abnormalities. Mol Cell Endocrinol, 225:101, 2004.

Imgrund, M., Grone, E., Grone, H. J., Kretzler, M., Holzman, L., Schlondorff, D., Rothenpieler, U. W.: Re-expression of the developmental gene Pax-2 during experimental acute tubular necrosis in mice. Kidney Int, 56:1423, 1999.

Maeshima, A., Maeshima, K., Nojima, Y., and Kojima, I.: Involvement (>f Pax-2 in the action of activin A on tubular cell regeneration. J Am Soc Nephrol, 13:2850, 2002.

Bolton, C. H., Downs, L. G., Victory, J. G., Dwight, J. F., Tomson, C. R., Mackness, M. I.: Endothelial dysfunction in chronic renal failure: roles of lipoprotein oxidation and pro-inflammatory cytokines. Nephrol Dial Transplant, 16:1189, 2001.

Singer, K. H.: Interactions between epithelial cells and T lymphocytes: role of adhesion molecules. J. Leukoc Biol, 48: 367, 1990.

Mason, J., Joeris, B., Welsch, J., and Kriz, W.: Vascular congestion in ischemic renal failure: the role of cell swelling. Miner Electrolyte Metab, 15:114, 1989

Olof, P., Hellberg, A., Kallskog, O., and Wolgast, M.: Red cell trapping and postischemic renal blood flow. Differences between the cortex, outer and inner medulla. Kidney Int, 40:625, 1991.

Kelly, K., Williams, W., Colvin, R., Meehan, S., Q. et al.: Intercellular Adhesion Molecule-1-deficient Mice Are Protected against Ischemic Renal Injury. Journal of Clin Inv, 97:1056, 1996.

Haller, H., Maasch, C., Dragun, D., Wellner, M., Q. et al.: Antisense Oligodeoxynucleotide Strategies in Renal and Cardiovascular Disease. Kidney Int, 53: 1550, 1998

Chen, W., Bennett, C.; Wang, M., Dragun, D. Q. et al.: Perfusion of kidneys with unformulated “naked” intercellular adhesion molecule-1 antisense oligodeoxynucleotides prevents ischemic/reperfusion injury. Transplantation, 68:880, 1999.

Dragun, D., Tulius, S., Park, J., Maasch, C., Q. et al. ICAM-1 antisense oligodeoxynucleotides prevent reperfusion injury and enhance immediate graft function in renal transplantation. Kidney Int, 54:590, 1998.

Feeley, B., Poston, R., Park, A., Ennen, M., Q. et al. Optimization of ex vivo pressure mediated delivery of antisense oligodeoxynucleotides to ICAM-1 reduces reperfusion injury in rat cardiac allografts. Transplantation, 69:1067, 2000

Chen, W., Bennett, F., Wang, M., Dragun, D., Q. et al.: Perfusion of kidneys with unformulated “naked” intercellular adhesion molecule-1 antisense oligodeoxynucleotides prevents ischemic/reperfusion injury. Transplantation, 68:880, 1997.

Stepkowski, S., Wang, M., Condon, T., Cheng-Flournoy, S., Q. et al: Protection against allograft rejection with intracellular adhesion molecule-1 antisense oligodeoxynucleotides. Transplantation, 66:699, 1998.

Kausch, I., and Bohle, A.: Antisense oligonucleotide therapy in urology. J Urol, 168:239, 2002.

Pouton, C. W., and Seymour, L. W.: Key issues in non-viral gene delivery. Adv Drug Rev., 46:187, 2001.

Ritter T., Kupiec-Weglinski, J. W.: Gene therapy for the prevention of ischemia/reperfusion injury in organ transplantation. Curr Gene Ther, 5:101, 2005.

Deglon, N., Hantraye, P.: Viral vectors as tools to model and treat neurodegenerative disorders. J. Gene Med:2005.

Shoji, Y., Nakashima, H. Current status of delivery systems to improve target efficacy of oligonucleotides. Curr Pharm Des, 7:785, 2004.

Brown, M. D., Schatzlein, A. G., Uchegbu, I. F.: Gene delivery with synthetic (non viral) carriers. Int J Pharm, 229:1, 2001.

Gao, X., Huang, L. Cationic liposome-mediated gene transfer. Gene Ther, 2:710, 1995.

Weyermann, J., Lochmann, D., Zimmer, A. Comparison of antisense oligonucleotide drug delivery systems. J. Control Release, 100:411, 2004.

Zhang, S., Xu, Y., Wang, B., Qiao, W., Liu, D., Li, Z. Cationic compounds used in lipoplexes and polyplexes for gene delivery. J. Control Release, 100:165, 2004.

Miller, A. D.: Human gene therapy comes of age. Nature, 357:455, 1992.

Galanis, E., Vile, R., Russell, S. J. Delivery systems intended for in vivo gene therapy of cancer: targeting and replication competent viral vectors. Crit Rev Oncol Hematol, 38:177, 2001.

Nakamura, N., Timmermann, S. A., Hart, D. A., Kaneda, Y., Shrive, N. G., et al.: A comparison of in vivo gene delivery methods for antisense therapy in ligament healing. Gene Ther, 5:1455, 1998.

Ledley, F. D. Nonviral gene therapy: the promise of genes as pharmaceutical products. Hum Gene Ther, 6:1129, 1995.

Fiset, P. O., Gounni, A. S.: Antisense oligonucleotides: problems with use and solutions. Reviews in Biology and Biotechnology, 1:27, 2001.

Takakura, Y., Mahato, R. I., Hashida, M.: Extravasation of macromolecules. Adv Drug Deliv Rev, 34:93, 1998.

Bally, M. B., Harvi, P., Wong, F. M., Kong, S., Q. et al.: Biological barriers to cellular delivery of lipid-based DNA carriers. Adv Drug Deliv Rev, 38:291, 1999.

Marwick, C.: First “antisense” drug will treat CMV retinitis. J of the Amer Med Assoc, 280:871, 1998.

Thomas, C., Ehrhardt, A., Kay, M. Progress and problems with the use of viral vectors for gene therapy. Nature Genetics, 4:346, 2003.

Yokoyama, M.: Gene delivery using temperature-responsive polymeric carriers. Drug Disc Today, 7:426, 2002.

Hinrichs, W. L., Schuurmans-Nieuwenbroek, N. M., Van de Wetering, P., Hennink, W. E.: Thermosensitive polymers as carriers for DNA delivery. J Cont Release, 60:249, 1999.

Saunders, B. R., and Vincent, B., Microgel particles as model colloids: theory, properties and applications. Advances in Colloid and Interface Science, 80:1, 1999.

Kurisawa, M., Yokoyama, M., and Okano, T.: Gene expression control by temperature with thermo-responsive polymeric gene carriers. J Cont Release, 69:127, 2000.

Murata, M., Kaku, W., Anada, T., Sato, Y.; Q. et al.: Novel DNA/Polymer conjugate for intelligent antisense reagent with improved nuclease resistance. Bioorganic & Medicinal Chemistry Letters, 17: 3967, 2003.

Oupicky, D., Konak, C., Ulbrich, K., Wolfert, M. A., Q. et al.: DNA delivery systems based on complexes of DNA with synthetic polycations and their copolymers. J of Cont Rel, 65:149, 2000.

Choksakulnimitr, S., Masuda, S., Tokuda, H., Takakura, Y., Q. et al.: In vitro cytotoxicity of macromolecules in different cell culture systems. J Cont Release, 34: 233, 1995.

Lien, Y. H., and Scott, K.: Long-term cyclophosphamide treatment for recurrent type I membranoproliferative glomerulonephritis after transplantation. Am J Kidney Dis, 35:539, 2000.

Lien, Y. H., Lai, L. W., and Silva, A. L.: Pathogenesis of renal ischemia/reperfusion injury: lessons from knockout mice. Life Sci, 74:543, 2003.

Lien, Y. H., Lai, L. W.: Gene therapy for renal disorders. Expert Opin Biol Ther, 4:919, 2004.

Lien, Y. H., and Lai, L.: Renal gene transfer: nonviral approaches. Mol. Biotech, 24: 283, 2003.

Lai, L. W., Erickson, R. P., Venta, P. J., Tashian, R. E., et al. Promoter activity of carbonic anhydrase II regulatory regions in cultured renal proximal tubular cells. Life Sci, 63:121, 1998.

Lai, L. W., Moeckel, G. W., Lien, Y. H. Kidney-targeted liposome-mediated gene transfer in mice. Gene Ther, 4:426, 1997.

Ramakumar, S., Roberts, W. W., Fugita, O. E., Colegrove, P., Q., et al.: Local hemostasis during laparoscopic partial nephrectomy using biodegradable hydrogels: initial porcine results. J Endourol, 16:489, 2002.

Park, E. L., Ulreich, J. B., Scott, K. M., Ramakumar, S. Evaluation of polyethylene glycol based hydrogel for tissue sealing after laparoscopic partial nephrectomy in a porcine model. J Urol, 172:2446, 2004.

Ramakumar, S., Phull, H., Purves, T., Funk, J., Lien., Yeong-Hau., et al.: Novel delivery of oligonucleotides using a topical hydrogel tissue sealant in a murine partial nephrectomy model. J Urol, pending.

Sharon, D. Crisman, M., Jonathan, R., Diamond, L., et al.: Angiotensinogen and AT1 antisense inhibition of osteopontin translation in rat proximal tubular cells. Am J Physiol Renal Physiol, 278:708, 2000.

Combe, C., Burton, C. J., Dufourco, P., Weston, S., et al.: Hypoxia induces intercellular adhesion molecule-1 on cultured human tubular cells. Kidney Int, 51:1703, 1997.

Maeshima, A., Nojima, Y., Kojima, I.: Activin A: an autocrine regulator of cell growth and differentiation in renal proximal tubular cells. Kidney Int, 62:446, 2002.

Kumar, Y., and Tatu, U. “Induced hsp70 is in small, cytoplasmic complexes in a cell culture model of renal ischemia: a comparative study with heat shock.” Cell Stress Chaperones, 5:314, 2000.

Paller, M. S., Patten, M. Protective effects of glutathione, glycine, or alanine in an in vitro model of renal anoxia. J Am Soc Nephrol, 2:1338, 1992.

Ferrari, M., Fornasiero, M. C., Isetta, A. M. MTT colorimetric assay for testing macrophage cytotoxic activity in vitro. J Immunol Methods, 131:165, 1990.

Aksoy, Y., Yapanoglu, T., Aksou, H., Yildirim, A. K. The effect of dehydroepiandrosterone on renal ischemia-reperfusion-induced oxidative stress in rabbits. Urol Res, 32:93, 2004.

Yoshida, R., Sakai, K., Okano, T., Sakurai, Y. Modulating the phase transition temperature and thermosensitivity in N-isopropylacrylamide copolymer gels. J Biomater Sci Polym Ed, 6:585,1994.

Eeckman, F., Moes, A. J., Amighi, K. Poly(N-isopropylacrylamide) copolymers for constant temperature controlled drug delivery. Int J Pharm, 273:109, 2004.

Kuroda, S. J-aggregation and its characterization in Langmuir-Blodgett films of merocyanine dyes. Adv Colloid Interface Sci, 111:181, 2004.

Ikegami, K. Spectroscopic study of J aggregates of amphiphilic merocyanine dyes formed in their pure Langmuir films. J Chem Phys, 121:2337, 2004.

Tachibana, H., Yamanaka, Y., Sakai, H., Matsumoto, M. et al.: J-Aggregate formation and morphological change on UV irradiation of the langmuir-blodgett films of spiropyran. Mol. Cryst. Liq. Cryst, 345:149, 2000.

Laxmikant, K., Schulten, K., et al.: NAMD2: Greater scalability for parallel molecular dynamics. Journal of Computational Physics, 151:283, 1999.

Marc, P., Ann Kuhn, M., Mehta, V., Besner, G.: Nanogels for oligonucleotide delivery to the brain. Bioconjugate Chem, 15:50, 2004.

McAllister, K., Sazani. P., Adam, M., Cho, M. J., Rubinstein, M., Samulski, R. J., DeSimone, J. M. Polymeric nanogels produced via inverse microemulsion polymerization as potential gene and antisense delivery agents. J Am Chem Soc, 124:15198, 2002.

Henry, S. P., Taylor, J., Midgley, L., et al.: Evaluation of the toxicity of ISIS 2302, a phosphorothioate oligonucleotide, in a 4-week study in CD-1 mice. Antisense Nucleic Acid Drug Dev, 7:473, 1997.

Monteith, D. K., Homer, M. J., Gillett, N. A., et al.: Evaluation of the renal effects of an antisense phosphorothioate oligodeoxynucleotide in monkeys. Toxicol. Pathol. 27:307, 1999.

Weinreich, T., Wuthrich, R. P., Booy, C., Binswanger, U. Suppression of ICAM-1 expression in renal proximal tubular cells by 1,25-dihydroxyvitamin D3. Kidney Blood Press Res, 24:92, 2001.

Junge, W., Wilke, B., Halabi, A., Klein, G. Determination of reference intervals for serum creatinine, creatinine excretion and creatinine clearance with an enzymatic and a modified Jaffe method. Clin Chim Acta, 344:137, 2004.

Weng, H., Zhou, J., Hu, B.: Tissue responses to thermally-responsive hydrogel nanoparticles. J. Biomater. Sci., Polymer Ed., 15:1167, 2004.

Geary, R. S., Yu, R. Z. and Levin, A. A. Pharmacokinetics of phosphorothioate antisense oligodeoxynucleotides. Curr. Opin. Investig. Drugs 2:562, 2001.

Vinogradov, S. V., Batrakova, E. V., Kabanov, A. V.: Pharmacology and toxicology of phosphorothioate oligonucleotides in the mouse, rat, monkey and man. Toxicol Lett, 83:425, 1995.

Ledoan, T., Auger, R., Benjahad, A., Tenu, J. P. “High specific radioactivity labeling of oligonucleotides with 3H-succinimidyl propionate.” Nucleosides Nucleotides 18:277, 1999.

Srinivasan, S. K., Tewary, H. K. and Iversen, P. L. Characterization of binding sites, extent of binding, and drug interactions of oligonucleotides with albumin. Antisense Res. Dev. 5:131, 1995.

Sawai, K., Miyao, T., Takakura, Y. and Hashida, M. “Renal disposition characteristics of oligonucleotides modified at terminal linkages in the perfused rat kidney.” Antisense Res. Dev. 5:279 (1995).

T. Tanaka, Phys. Rev. Lett. 40:820 (1978).

A. Suzuki and T. Tanaka, “Phase Transition in Polymer Gels Induced by Visible Light,” Nature 346:345-347 (1990).

M. Irie and D. Kunwatchakun, Macrom. Rapid Comm. pp 2476-2480.

A. Mamada, T. Tanaka, D. Kungwatchakun, M. Irie, Macromolecules 23:1517 (1990).

X. M. Zhang, Y. Li, Z. B. Hu, C. L. Littler, J. Chem. Phys. 102:551 (1995).

R. Akashi, H. Tsutsui, and A. Komura, Adv. Mat. 14:1808 (2002).

T. Ikeda, M. Nakano, Y. Yu, O. Tsutsumi, and A. Kanazawa, Adv. Mater. 15:201 (2003).

T. Hirakura, Y. Nomura, Y. Aoyama, K. Akiyoshi, Biomacromolecules 5(5):1804-1809 (2004).

K. Sumaru, M. Kameda, T. Kanamori, T. Shinbo Macromolecules 37:4949-4955 (2004).

M. Kameda, K. Sumaru, T. Kanamori, T. Shinbo, Langmuir 20(21):9315-9319 (2004).

K. Sumaru, M. Kameda, T. Kanamori, T. Shinbo Macromolecules 37:7854-7856 (2004).

1. A composition produced by the process comprising polymerizing a hydrogel precursor with a spiropyran.

2. The composition of claim 1, wherein the hydrogel precursor comprises a compound having at least one alkenyl group.

3. The composition of claim 1, wherein the hydrogel precursor comprises acrylonitrile, acrylic acid, acrylamide, or methacrylic acid.

4. The composition of claim 1, wherein the hydrogel precursor comprises a substituted acrylamide.

5. The composition of claim 1, wherein the hydrogel precursor comprises an N-alkyl substituted acrylamide.

6. The composition of claim 1, wherein the hydrogel precursor comprises N-methylacrylamide, N-ethylacrylamide, N-propyllacrylamide, or N-isopropylacrylamide.

7. The composition of claim 1, wherein the spiropyran comprises at least one alkenyl group.

8. The composition of claim 1, wherein the spiropyran comprises the Formula I. 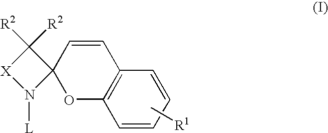
wherein,
X is a substituted or unsubstituted, C1 to C4, alkyl or alkenyl group;
R¹ is H, alkyl, alkenyl, alkoxy, aryl, halide, hydroxyl, amino, nitro, silyl, sulfo-oxo, sulfonylamino, ether, ester, carboxylic acid, or thiol group;
each R² is, independently of each other, H, alkyl, alkenyl, alkoxy, aryl, halide, hydroxyl, amino, nitro, silyl, sulfo-oxo, sulfonylamino, thiol, ether, ester, carboxylic acid, or together each R² substituent forms a keto group, a cyclicalkyl group, a cyclicalkenyl group, or an aryl group; and
L comprises an alkenyl group.

9. The composition of claim 7, wherein X is a fused aryl group.

10. The composition of claim 9, wherein each R² is an alkyl group.

11. The composition of claim 10, wherein R¹ is NO₂.

12. The composition of claim 1, wherein the spiropyran has the Formula II. 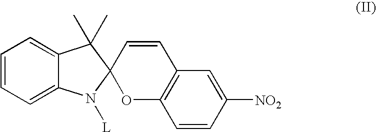
wherein L is —(CH₂)ₘC(O)NH(CH₂)ₙCH═CH₂, wherein m is from 1 to 12 and n is from 0 to 12.

13. The composition of claim 12, wherein m is 3 and n is 1.

14. The composition of claim 1, wherein the process further comprises the addition of a crosslinking agent.

15. The hydrogel of claim 14, wherein the crosslinking agent comprises a compound comprising at least two alkenyl groups.

16. The composition of claim 14, wherein the crosslinking agent comprises N,N′-methylene-bis-acrylamide.

17. A composition produced by the process comprising reacting a hydrogel precursor comprising at least one hydroxyl group and/or carboxylic acid group with a spiropyran comprising a group capable of reacting with the hydroxyl group or carboxylic acid group.

18. The composition of claim 17, wherein the hydrogel precursor comprises hydroxypropylcellulose or hyaluronic acid.

19. The composition of claim 17, wherein the hydrogel precursor is polymerized in the absence of a surfactant.

20. (canceled)

21. A composition comprising a hydrogel and a spiropyran, wherein the spiropyran is bonded to the hydrogel.

22. The composition of claim 21, wherein the hydrogel is present in an amount of from about 99 to about 80 weight percent and the spiropyran is present in an amount of from about 1 to about 20 weight percent.

23. The composition of claim 21, wherein the composition comprises a microgel.

24. The composition of claim 21, wherein the composition comprises a nanogel.

25. The composition of claim 21, wherein the composition comprises a colloidosome.

26. The composition of claim 21, wherein the composition decreases in size upon exposure to UV light.

27. The composition of claim 21, wherein the composition increases in size upon exposure to visible light.

28. A pharmaceutical formulation composition comprising the composition of claim 21 and a pharmaceutical carrier.

29. The pharmaceutical formulation of claim 27, further comprising a pharmaceutical active.

30. The pharmaceutical formulation of claim 28, wherein the pharmaceutical active comprises a cell.

31. The pharmaceutical formulation of claim 28, wherein the pharmaceutical active comprises a nucleic acid.

32. The pharmaceutical formulation of claim 28, wherein the pharmaceutical active is an antisence oligonucleotide.

33. A method of delivering a pharmaceutical active to a subject, comprising administering the composition of claim 21 and a pharmaceutical active.

34. The method of claim 33, wherein the pharmaceutical active comprises a nucleic acid.

35. A method of decreasing an inflammatory response in a subject comprising administering the composition of claim 21 and an antisense oligonucleotide of ICAM-1.
